(19) United States
(12) Patent Application Publication
(10) Pub. No.: US 2015/0174095 A1
(43) Pub. Date: Jun. 25, 2015

(54) PHARMACEUTICAL COMPOSITIONS FOR THE TREATMENT OF VISUAL SYSTEM DISORDERS

(71) Applicant: Allergan, Inc., Irvine, CA (US)

(72) Inventors: Alan C. Foster, San Diego, CA (US); Yong-Xin Li, Mission Viejo, CA (US); Ursula V. Staubli, Laguna Beach, CA (US)

(21) Appl. No.: 14/581,272

(22) Filed: Dec. 23, 2014

Related U.S. Application Data
(60) Provisional application No. 61/919,964, filed on Dec. 23, 2013.

(51) Int. Cl. A61K 31/401; A61K 9/00

(52) U.S. Cl. CPC A61K 31/401; A61K 9/0048

(57) ABSTRACT

The present invention relates to pharmaceutical compositions comprising D-serine transporter inhibitors which are proline analogues and therapeutic methods using such pharmaceutical compositions in methods for the treatment of visual system disorders and the enhancement of the visual function.

### CROSS-REFERENCE TO RELATED APPLICATIONS

The present application claims the benefit of U.S. provisional application 61/919,964 entitled “Pharmaceutical Compositions For The Treatment Of Visual System Disorders” and having docket number 19353PROV (AP) filed on Dec. 23, 2013, which is incorporated herein by reference in its entirety and serves as the basis for a priority and/or benefit claim for the present application.

### FIELD OF THE INVENTION

The present invention relates to pharmaceutical compositions comprising D-serine transporter inhibitors which are proline analogues and therapeutic methods using such pharmaceutical compositions in methods for the treatment of visual system disorders and the enhancement of the visual function.

### BACKGROUND

Neuronal circuits in the central nervous system rely on the release of chemical neurotransmitters from specialized connections called synapses for communication. The major excitatory neurotransmitter is the amino acid glutamate, and release of glutamate from a pre-synaptic terminal elicits a response through activation of several types of receptors. One of the sub-types of glutamate receptors, the N-methyl-D-aspartate (NMDA) receptor, plays a major role in neuronal communication and in the plasticity of synaptic responses that occurs under both physiological and pathophysiological conditions.

NMDA receptors are ligand-gated cation channels comprised of a tetrameric assembly of NR1, NR2 and NR3 sub-units (Paoletti and Neyton, 2007). They are unique amongst neurotransmitter receptors in that they require occupation of two separate recognition sites for activation. An acidic amino acid site where glutamate binds, is located on the NR2 sub-units, and a neutral amino acid (or co-agonist) site is located on the NR1 sub-unit. The endogenous co-agonist for this site was originally thought to be glycine, but more recent evidence indicated that D-serine is also an endogenous co-agonist. In fact, in higher brain regions D-serine may be the dominant co-agonist. Occupation of the co-agonist site is essential for glutamate (or a glutamate analog) to activate the NMDA receptor, and in native assays the removal of glycine or D-serine by exogenously-applied degradative enzymes can reduce or abolish NMDA receptor-mediated responses. For example, in the rat hippocampal slice, application of the D-serine metabolizing enzyme, D-amino acid oxidase (D-AAO), completely prevents the induction of long-term potentiation (LTP) a form of synaptic plasticity whose initiation is dependent on NMDA receptor activation (Yang et al., 2003). This suggests that the dominant co-agonist in this case is D-serine, since glycine is not a substrate for D-AAO.

The mechanisms that regulate extracellular D-serine, and therefore govern how NMDA receptors are activated, are not well understood. In keeping with other neurotransmitters and neuromodulators, it is likely that transporters on the cell surface are involved in regulating synaptic levels of D-serine. Amino acid transporters usually prefer L-amino acids, however D-serine has been shown to be a substrate for certain transporters. These include the heterodimeric transporter asc-1 (SLC3A2/SLC7A10) which has micromolar affinity for D-serine, ASCT2 (SLC1A5), ATB⁰⁺ (SLC7A9) and PAT1-4 (SLC36A1-4). Based on the tissue and cellular localization, the primary candidates for transporters that regulate synaptic D-serine levels are asc-1 (neuronal) and ASCT2 (glial). The related transporter ASCT1 (SLC1A4) also has been localized to neurons and glia, however it has been reported that D-serine is not a substrate for ASCT1 (Shafqat et al., 1993). None of these transporters are selective for D-serine, and their substrates are typically small neutral amino acids such as serine, alanine, cysteine and threonine. They also are known to function as exchangers that can flux their substrates both into and out of cells. Consequently, it has been unclear if these transporters are responsible primarily for the net uptake or the net release of D-serine and other substrates. Considering that asc-1 has the highest known affinity for D-serine, it has been thought that this transporter is primarily responsible for removing D-serine from the extracellular space (Rutter et al., 2007). In support of this, the asc-1 knock-out mouse has a phenotype that includes increased excitability (Xie et al., 2005).

In the visual system, NMDA receptors are important mediators of glutamate-mediated neurotransmission and synaptic plasticity. This occurs at all levels of the visual axis, including neurons in the retina, in the central neurons that receive retinal ganglion cell input in the lateral geniculate nucleus and the superior colliculus, and in the visual cortex. Based on experiments using D-AAO, D-serine has been shown to be an endogenous co-agonist involved in NMDA-receptor-mediated synaptic responses in the retina (Stevens et al., 2003) NMDA receptors have also been shown to mediate synaptic responses in the lateral geniculate (Harveit & Heggelund, 1990; Scharfman et al., 1990) and the visual cortex (ie the primary pathways that transduce visual information). In the visual cortex, NMDA receptors mediate the phenomenon of long-term potentiation (LTP), an important form of synaptic plasticity. NMDA receptor-dependent LTP occurs in many brain regions and is viewed as a mechanism of synaptic strengthening that is fundamental to the establishment and maintenance of appropriate synaptic connections. In the hippocampus, for example, LTP has been studied as a synaptic surrogate of learning and memory. In visual cortex neurons, LTP mediates stimulus-specific response potentiation, a form of experience-dependent plasticity that contributes to visual function (Cooke and Bear, 2010).

In retinal diseases such as glaucoma and macular degeneration, loss of the vision arises from degeneration or malfunction of retinal cells. Consequently, the normal neuronal transmission along the visual pathway is disrupted in the affected parts of the visual field. One strategy to remedy this loss of function would be to enhance the visual neurotransmission that remains unaffected by disease to compensate for the region of impairment. In addition, enhancing the plasticity of neuronal connections that occurs in the adult visual system could lead to the establishment of new neuronal connections that replace the lost function and improve visual performance.

Enhancing NMDA receptor activity by increasing the extracellular levels of D-serine would boost visual performance and compensate for the loss of vision resulting from retinal disease. As a result, we have discovered compounds that inhibit the transport of D-serine and enhance NMDA receptor-mediated synaptic responses. We identified the D-serine transporters that are important for regulating NMDA receptor-mediated LTP in the visual cortex, and we demonstrated that D-serine transport inhibitors improve visual function in animal models of retinal disease.

### SUMMARY OF THE INVENTION

The present invention relates to the use of pharmaceutical compositions comprising D-serine transporter inhibitor compound(s) in methods for the treatment of visual system disorders.

Visual system disorders which may be treated with the D-serine transport inhibitors include macular edema, dry and wet macular degeneration, choroidal neovascularization, diabetic retinopathy, acute macular neuroretinopathy, central serous chorioretinopathy, cystoid macular edema, and diabetic macular edema, uveitis, retinitis, choroiditis, acute multifocal placoid pigment epitheliopathy, Behcet's disease, birdshot retinochoroidopathy, syphilis, lyme, tuberculosis, toxoplasmosis, intermediate uveitis (pars planitis), multifocal choroiditis, multiple evanescent white dot syndrome (mewds), ocular sarcoidosis, posterior scleritis, serpiginous choroiditis, subretinal fibrosis and uveitis syndrome, Vogt-Koyanagi—and Harada syndrome; retinal arterial occlusive disease, anterior uveitis, retinal vein occlusion, central retinal vein occlusion, disseminated intravascular coagulopathy, branch retinal vein occlusion, hypertensive fundus changes, ocular ischemic syndrome, retinal arterial microaneurysms, Coat's disease, parafoveal telangiectasis, hemiretinal vein occlusion, papillophlebitis, central retinal artery occlusion, branch retinal artery occlusion, carotid artery disease (CAD), frosted branch angiitis, sickle cell retinopathy, angioid streaks, familial exudative vitreoretinopathy, and Eales disease; traumatic/surgical conditions such as sympathetic ophthalmia, uveitic retinal disease, retinal detachment, trauma, photocoagulation, hypoperfusion during surgery, radiation retinopathy, and bone marrow transplant retinopathy; proliferative vitreal retinopathy and epiretinal membranes, and proliferative diabetic retinopathy; infectious disorders such as ocular histoplasmosis, ocular toxocariasis, presumed ocular histoplasmosis syndrome (PONS), endophthalmitis, toxoplasmosis, retinal diseases associated with HIV infection, choroidal disease associate with HIV infection, uveitic disease associate with HIV infection, viral retinitis, acute retinal necrosis, progressive outer retinal necrosis, fungal retinal diseases, ocular syphilis, ocular tuberculosis, diffuse unilateral subacute neuroretinitis, and myiasis; genetic disorders such as retinitis pigmentosa, systemic disorders with associated retinal dystrophies, congenital stationary night blindness, cone dystrophies, Stargardt's disease and fundus flavimaculatus, Best's disease, pattern dystrophy of the retinal pigmented epithelium, X-linked retinoschisis, Sorsby's fundus dystrophy, benign concentric maculopathy, Bietti's crystalline dystrophy, and pseudoxanthoma elasticum; retinal tears/holes such as retinal detachment, macular hole, and giant retinal tear; tumors such as retinal disease associated with tumors, congenital hypertrophy of the retinal pigmented epithelium, posterior uveal melanoma, choroidal hemangioma, choroidal osteoma, choroidal metastasis, combined hamartoma of the retina and retinal pigmented epithelium, retinoblastoma, vasoproliferative tumors of the ocular fundus, retinal astrocytoma, and intraocular lymphoid tumors; punctate inner choroidopathy, acute posterior multifocal placoid pigment epitheliopathy, myopic retinal degeneration, acute retinal pigment epitheliitis, retinitis pigmentosa, proliferative vitreal retinopathy (PVR), age-related macular degeneration (ARMD), diabetic retinopathy, diabetic macular edema, retinal detachment, retinal tear, uveitis, cytomegalovirus retinitis, glaucoma, amblyopia, stroke-induced blindness, visual dysfunction in Parkinson's disease, Alzheimer's disease and multiple sclerosis, seizure-induced cortical blindness, induced visual dysfunction, and epileptic blindness.

US Patent Application Publication No. 2012/0302621 A1 and US Patent Application Publication No. 2012/0329873 A1, disclose numerous in vivo and in vitro models, demonstrating that elevation of D-serine levels in the visual system can improve visual function both in the normal state and when vision is impaired by retinal dysfunction. Furthermore, US Patent Application Publication No. 2012/0302621 A1 and US Patent Application Publication No. 2012/0329873 A1 show that inhibiting the neutral amino acid transporters ASCT1 and ASCT2, for which D-serine is a substrate, also leads to the improvement of visual function in the normal and retinal dysfunction states through elevation of endogenous D-serine.

US Patent Application Publication No. 2012/0302621A1 teaches that L-4-trans-hydroxyproline and related benzylproline analogs are inhibitors of ASCT1 and ASCT2, and improve visual function in an in vitro model.

Elevation of D-serine is also desirable as a treatment for CNS disorders where a deficit in NMDA receptor function occurs. D-serine transport inhibitors may be used to treat schizophrenia, schizophreniform disorder, and schizoaffective disorder and specifically conditions selected from the following: conduct disorder, solitary aggressive type conduct disorder, undifferentiated type Tourette's disorder chronic motor or vocal tic disorder, transient tic disorder, alcohol withdrawal delirium, alcohol hallucinosis, alcohol dementia associated with alcoholism, amphetamine or similarly acting sympathomimetic intoxication, amphetamine or similarly acting sympathomimetic delirium, amphetamine or similarly acting sympathomimetic delusional disorder, cannabis delusional disorder, cocaine intoxication, cocaine delirium, cocaine delusional disorder, hallucinogen hallucinosis, hallucinogen delusional disorder, hallucinogen mood disorder, hallucinogen post-hallucinogen perception disorder, phencyclidine or similarly acting arylcyclohexylamine intoxication, phencyclidine or similarly acting arylcyclohexylamine delirium, phencyclidine or similarly acting arylcyclohexylamine delusional disorder, phencyclidine or similarly acting arylcyclohexylamine mood disorder, phencyclidine or similarly acting arylcyclohexyilamine organic mental disorder, other or unspecified psychoactive substance intoxication, other or unspecified psychoactive substance delirium, other or unspecified psychoactive substance dementia, other or unspecified psychoactive substance delusional disorder, other or unspecified psychoactive substance hallucinosis, other or unspecified psychoactive substance mood disorder, other or unspecified psychoactive substance anxiety disorder, other or unspecified psychoactive substance personality disorder, other or unspecified psychoactive substance organic mental disorder, delirium, dementia, organic delusional disorder, organic hallucinosis, organic mood disorder, organic anxiety disorder, organic personality disorder, organic mental disorder, obsessive compulsive disorder, post-traumatic stress disorder, generalized anxiety disorder, anxiety disorder, body dysmorphic disorder, hypo-chondriasis (or hypochondriacal neurosis), somatization disorder, undifferentiated somatoform disorder, somatoform disorder, intermittent explosive disorder, kleptomania, pathological gambling, pyromania, trichotillomania, and impulse control disorder, schizophrenia, catatonic, subchronic, schizophrenia, catatonic, chronic, schizophrenia, catatonic, sub chronic with acute exacerbation, schizophrenia, catatonic, chronic, with acute exacerbation, schizophrenia, catatonic, in remission, schizophrenia, catatonic, unspecified, schizophrenia, disorganized, subchronic, schizophrenia, disorganized, chronic, schizophrenia, disorganized, subchronic with acute exacerbation, schizophrenia, disorganized, chronic with acute exacerbation, schizophrenia, disorganized, in remission, schizophrenia, disorganized, unspecified, schizophrenia, paranoid, subchronic, schizophrenia, paranoid, chronic, schizophrenia, paranoid, sub chronic with acute exacerbation, schizophrenia, paranoid, chronic with acute exacerbation, schizophrenia, paranoid, in remission, schizophrenia, paranoid, unspecified, schizophrenia, undifferentiated, subchronic, schizophrenia, undifferentiated, chronic, schizophrenia, undifferentiated, sub chronic with acute exacerbation, schizophrenia, undifferentiated, chronic with acute exacerbation, schizophrenia, undifferentiated, in remission, schizophrenia, undifferentiated, unspecified, schizophrenia, residual, subchronic, schizophrenia, residual, chronic, schizophrenia, residual, subchronic with acute exacerbation, schizophrenia, residual, chronic with acute exacerbation, schizophrenia residual in remission, schizophrenia, residual, subchronic schizophrenia, residual, chronic, schizophrenia, residual, subchronic with acute exacerbation, schizophrenia, residual, chronic with acute exacerbation, schizophrenia, residual, in remission, schizophrenia, residual, unspecified, delusional (paranoid) disorder, brief reactive psychosis, schizophreniform disorder, schizoaffective disorder, induced psychotic disorder, psychotic disorder (atypical psychosis), personality disorders, paranoid, personality disorders, schizoid, personality disorders, schizotypal, personality disorders, antisocial, and personality disorders.

D-serine transport inhibitors may be used to treat a patient suffering from one or more types of cognitive disorder, such as agnosia, amnesia, aphasia, apraxia, delirium, dementia, and a learning disorder.

### DESCRIPTION OF THE DRAWINGS

FIGS. 1A and 1B show the dose-dependent effect of (2R,3S,4S)-4-(biphenyl-4-ylmethoxy)-3-hydroxypyrrolidine-2-carboxylic acid on transport currents recorded on HEK293 cells stably expressing ASCT1 or ASCT2 transporters. FIG. 1A: without L-serine; FIG. 1B: with 0.3 mM L-serine. The dots/lines represent data from ASCT2 and squares/lines represent data from ASCT1. Each point represents MEAN±SE from 4 to 5 cells. The effects are normalized to those of 3 mM 4-hydroxyphenyglycine (100% of Max). IC₅₀ values in μM are listed.

FIGS. 2A and 2B show the dose-dependent effect of (2R,3S,4S)-3-hydroxy-4-[(3-phenoxybenzyl)oxy]pyrrolidine-2-carboxylic acid on transport currents recorded on HEK293 cells stably expressing ASCT1 or ASCT2 transporters. FIG. 2A: without L-serine; FIG. 2B: with 0.3 mM L-serine. The dots/lines represent data from ASCT2 and squares/lines represent data from ASCT1. Each point represents MEAN±SE from 4 to 5 cells. The effects are normalized to those of 3 mM 4-hydroxyphenyglycine (100% of Max). IC₅₀ values in μM are listed.

FIG. 3 represents a graph showing that (2R,3S,4S)-4-(biphenyl-4-ylmethoxy)-3-hydroxypyrrolidine-2-carboxylic acid dose-dependently facilitates long-term potentiation (LTP) in the primary visual cortex of rats at 0.1 and 0.3 μM.

### DETAILED DESCRIPTION OF THE INVENTION

In one aspect the invention relates to a method for the treatment of visual system disorders caused by a deficit in N-methyl-D-aspartate receptor function, the method comprising administering to a subject in need thereof an ophthalmically acceptable pharmaceutical composition containing a therapeutically effective amount of one or more D-serine transporter inhibitor compounds which are proline analogues.

In another aspect the invention relates to a method for the treatment of visual system disorders, the method comprising administering to a subject in need thereof an ophthalmically acceptable pharmaceutical composition containing a therapeutically effective amount of one or more proline analogues selected from Formula I,

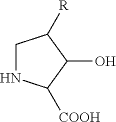

as disclosed in US Patent Application Publication No. 2013/0065935 A1.

In another aspect, the invention provides a method for the treatment of visual system disorders caused by a deficit in N-methyl-D-aspartate receptor function, the method comprising administering to a subject in need thereof an ophthalmically acceptable pharmaceutical composition containing a therapeutically effective amount of one or more D-serine transporter inhibitor compounds which are proline analogues selected from the group of compounds from Table 1, as disclosed in US Patent Application Publication No. 2013/0065935 A1.

In another aspect, the invention provides a method for the enhancement of the visual function, the method comprising administering to a subject in need thereof, an ophthalmically acceptable pharmaceutical composition containing a therapeutically effective amount of one or more D-serine transporter inhibitor compounds which are proline analogues selected from the group of compounds from Table 1, as disclosed in US Patent Application Publication No. 2013/0065935 A1.

TABLE 1

| Structure | name | |
|---|---|---|
| 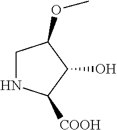 | Trans-3-hydroxy-cis-4-methoxy-L- proline | |
| 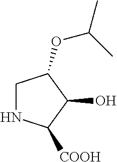 | Cis-3-hydroxy-trans-4- isopropoxy-L-proline | |
| 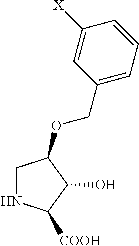 | H Cl F Br Me OMe | Trans-3-hydroxy-cis-4- benzyloxy-L-proline Trans-3-hydroxy-cis-4-(3- chlorobenzyloxy)-L- proline Trans-3-hydroxy-cis-4-(3- fluorobenzyloxy)-L-proline Trans-3-hydroxy-cis-4-(3- bromobenzyloxy)-L- proline Trans-3-hydroxy-cis-4-(3- methylbenzyloxy)-L- proline Trans-3-hydroxy-cis-4-(3- methoxybenzyloxy)-L- |
| | | proline |
| | Iso-Pr | Trans-3-hydroxy-cis-4-(3- |
| | | isopropylbenzyloxy)-L- |
| | | proline |
| | Ph | Trans-3-hydroxy-cis-4-(3- |
| | | phenylbenzyloxy)-L- |
| | | proline |
| | CF₃ | Trans-3-hydroxy-cis-4-(3- |
| | | trifluoromethylbenzyloxy)- |
| | | L-proline |
| 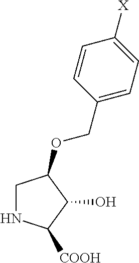 | Cl F Br Me OMe Iso-Pr | Trans-3-hydroxy-cis-4-(4- chlorobenzyloxy)-L- proline Trans-3-hydroxy-cis-4-(4- fluorobenzyloxy)-L-proline Trans-3-hydroxy-cis-4-(4- bromobenzyloxy)-L- proline Trans-3-hydroxy-cis-4-(4- methylbenzyloxy)-L- proline Trans-3-hydroxy-cis-4-(4- methoxybenzyloxy)-L- proline Trans-3-hydroxy-cis-4-(4- isopropylbenzyloxy)-L- |
| | | proline |
| | Ph | Trans-3-hydroxy-cis-4-(4- |
| | | phenylbenzyloxy)-L- |
| | | proline |
| | CF₃ | Trans-3-hydroxy-cis-4-(4- |
| | | trifluoromethylbenzyloxy)- |
| | | L-proline |
| 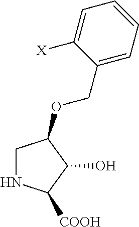 | Cl F Br Me OMe | Trans-3-hydroxy-cis-4-(2- chlorobenzyloxy)-L- proline Trans-3-hydroxy-cis-4-(2- fluorobenzyloxy)-L-proline Trans-3-hydroxy-cis-4-(2- bromobenzyloxy)-L- proline Trans-3-hydroxy-cis-4-(2- methylbenzyloxy)-L- proline Trans-3-hydroxy-cis-4-(2- methoxybenzyloxy)-L- proline |
| | Iso-Pr | Trans-3-hydroxy-cis-4-(2- |
| | | isopropylbenzyloxy)-L- |
| | | proline |
| | Ph | Trans-3-hydroxy-cis-4-(2- |
| | | phenylbenzyloxy)-L- |
| | | proline |
| | CF₃ | Trans-3-hydroxy-cis-4-(2- |
| | | trifluoromethylbenzyloxy)- |
| | | L-proline |
| 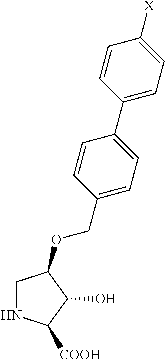 | Cl F Br Me OMe | (2R,3S,4S)-3-Hydroxy-4- ((4′-chlorol-[1,1′- biphenyl]-4- yl)methoxy)pyrrolidine-2- carboxylic acid (2R,3S,4S)-3-Hydroxy-4- ((4′-fluoro-[1,1′-biphenyl]- 4-yl)methoxy)pyrrolidine- 2-carboxylic acid (2R,3S,4S)-3-Hydroxy-4- ((4′-bromo-[1,1′-biphenyl]- 4-yl)methoxy)pyrrolidine- 2-carboxylic acid (2R,3S,4S)-3-Hydroxy-4- ((4′-methyl-[1,1′- biphenyl]-4- yl)methoxy)pyrrolidine-2- carboxylic acid (2R,3S,4S)-3-Hydroxy-4- ((4′-methoxy-[1,1′- biphenyl]-4- yl)methoxy)pyrrolidine-2- |
| | | carboxylic acid |
| | Iso-Pr | (2R,3S,4S)-3-Hydroxy-4- |
| | | ((4′-isopropyl-[1,1′- |
| | | biphenyl]-4- |
| | | yl)methoxy)pyrrolidine-2- |
| | | carboxylic acid |
| | Ph | (2R,3S,4S)-3-Hydroxy-4- |
| | | ((4′-phenyl-[1,1′- |
| | | biphenyl]-4- |
| | | yl)methoxy)pyrrolidine-2- |
| | | carboxylic acid |
| | CF₃ | (2R,3S,4S)-3-Hydroxy-4- |
| | | ((4′-trifluoromethyl-[1,1′- |
| | | biphenyl]-4- |
| | | yl)methoxy)pyrrolidine-2- |
| | | carboxylic acid |
| 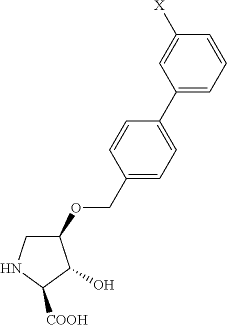 | Cl F Br Me OMe | (2R,3S,4S)-3-Hydroxy-4- ((3′-chlorol-[1,1′- biphenyl]-4- yl)methoxy)pyrrolidine-2- carboxylic acid (2R,3S,4S)-3-Hydroxy-4- ((3′-fluoro-[1,1′-biphenyl]- 4-yl)methoxy)pyrrolidine- 2-carboxylic acid (2R,3S,4S)-3-Hydroxy-4- ((3′-bromo-[1,1′-biphenyl]- 4-yl)methoxy)pyrrolidine- 2-carboxylic acid (2R,3S,4S)-3-Hydroxy-4- ((3′-methyl-[1,1′- biphenyl]-4- yl)methoxy)pyrrolidine-2- carboxylic acid (2R,3S,4S)-3-Hydroxy-4- ((3′-methoxy-[1,1′- |
| | | biphenyl]-4- |
| | | yl)methoxy)pyrrolidine-2- |
| | | carboxylic acid |
| | Iso-Pr | (2R,3S,4S)-3-Hydroxy-4- |
| | | ((3′-isopropyl-[1,1′- |
| | | biphenyl]-4- |
| | | yl)methoxy)pyrrolidine-2- |
| | | carboxylic acid |
| | Ph | (2R,3S,4S)-3-Hydroxy-4- |
| | | ((3′-phenyl-[1,1′- |
| | | biphenyl]-4- |
| | | yl)methoxy)pyrrolidine-2- |
| | | carboxylic acid |
| | CF₃ | (2R,3S,4S)-3-Hydroxy-4- |
| | | ((3′-trifluoromethyl-[1,1′- |
| | | biphenyl]-4- |
| | | yl)methoxy)pyrrolidine-2- |
| | | carboxylic acid |
| 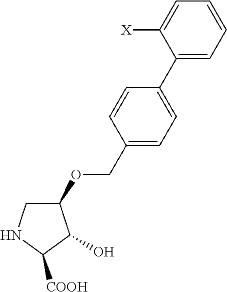 | Cl F Br Me | (2R,3S,4S)-3-Hydroxy-4- ((2′-chlorol-[1,1′- biphenyl]-4- yl)methoxy)pyrrolidine-2- carboxylic acid (2R,3S,4S)-3-Hydroxy-4- ((2′-fluoro-[1,1′-biphenyl]- 4-yl)methoxy)pyrrolidine- 2-carboxylic acid (2R,3S,4S)-3-Hydroxy-4- ((2′-bromo-[1,1′-biphenyl]- 4-yl)methoxy)pyrrolidine- 2-carboxylic acid (2R,3S,4S)-3-Hydroxy-4- ((2′-methyl-[1,1′- biphenyl]-4- yl)methoxy)pyrrolidine-2- carboxylic acid |
| | OMe | (2R,3S,4S)-3-Hydroxy-4- |
| | | ((2′-methoxy-[1,1′- |
| | | biphenyl]-4- |
| | | yl)methoxy)pyrrolidine-2- |
| | | carboxylic acid |
| | Iso-Pr | (2R,3S,4S)-3-Hydroxy-4- |
| | | ((2′-isopropyl-[1,1′- |
| | | biphenyl]-4- |
| | | yl)methoxy)pyrrolidine-2- |
| | | carboxylic acid |
| | Ph | (2R,3S,4S)-3-Hydroxy-4- |
| | | ((2′-phenyl-[1,1′- |
| | | biphenyl]-4- |
| | | yl)methoxy)pyrrolidine-2- |
| | | carboxylic acid |
| | CF₃ | (2R,3S,4S)-3-Hydroxy-4- |
| | | ((2′-trifluoromethyl-[1,1′- |
| | | biphenyl]-4- |
| | | yl)methoxy)pyrrolidine-2- |
| | | carboxylic acid |
| 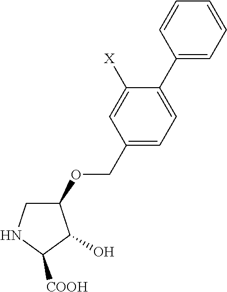 | Cl F Br Me OMe | (2R,3S,4S)-3-Hydroxy-4- ((2-chlorol-[1,1′-biphenyl]- 4-yl)methoxy)pyrrolidine- 2-carboxylic acid (2R,3S,4S)-3-Hydroxy-4- ((2-fluoro-[1,1′-biphenyl]- 4-yl)methoxy)pyrrolidine- 2-carboxylic acid (2R,3S,4S)-3-Hydroxy-4- ((2-bromo-[1,1′-biphenyl]- 4-yl)methoxy)pyrrolidine- 2-carboxylic acid (2R,3S,4S)-3-Hydroxy-4- ((2-methyl-[1,1′-biphenyl]- 4-yl)methoxy)pyrrolidine- 2-carboxylic acid (2R,3S,4S)-3-Hydroxy-4- ((2-methoxy-[1,1′- |
| | | biphenyl]-4- |
| | | yl)methoxy)pyrrolidine-2- |
| | | carboxylic acid |
| | Iso-Pr | (2R,3S,4S)-3-Hydroxy-4- |
| | | ((2-isopropyl-[1,1′- |
| | | biphenyl]-4- |
| | | yl)methoxy)pyrrolidine-2- |
| | | carboxylic acid |
| | Ph | (2R,3S,4S)-3-Hydroxy-4- |
| | | ((2-phenyl-[1,1′-biphenyl]- |
| | | 4-yl)methoxy)pyrrolidine- |
| | | 2-carboxylic acid |
| | CF₃ | (2R,3S,4S)-3-Hydroxy-4- |
| | | ((2-trifluoromethyl-[1,1′- |
| | | biphenyl]-4- |
| | | yl)methoxy)pyrrolidine-2- |
| | | carboxylic acid |

| | |
|---|---|
| 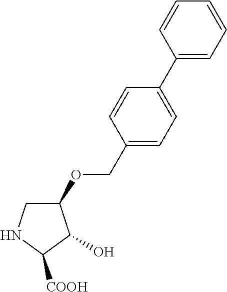 [SMILES: O=C(O)[C@H]1NC[C@@H](OCc2ccc(-c3ccccc3)cc2)[C@@H]1O] | (2R,3S,4S)-4-(Biphenyl-4- ylmethoxy)-3-hydroxypyrrolidine-2- carboxylic acid |
| 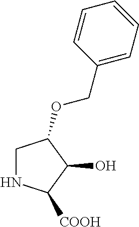 [SMILES: O=C(O)[C@H]1NC[C@H](OCc2ccccc2)[C@H]1O] | Cis-3-hydroxy-trans-4-benzyloxy-L- proline |
| 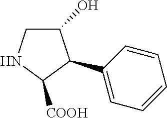 [SMILES: O=C(O)[C@H]1NC[C@H](O)[C@H]1c1ccccc1] | (2S,3R,4R)-3-Hydroxy-4-phenyl- proline |
| 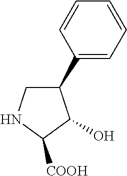 [SMILES: O=C(O)[C@H]1NC[C@@H](c2ccccc2)[C@@H]1O] | (2S,3S,4R)-3-Phenyl-4-hydroxy- proline |
| 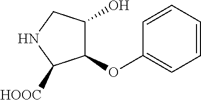 [SMILES: O=C(O)[C@H]1NC[C@H](O)[C@H]1Oc1ccccc1] | (2S,3S,4R)-3-Hydroxy-4- phenoxy-proline |
| 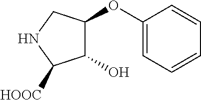 [SMILES: O=C(O)[C@H]1NC[C@@H](Oc2ccccc2)[C@@H]1O] | (2S,3S,4S)-3-Phenoxy-4- hydroxy-proline |
| 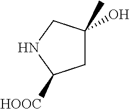 | Cis-4-methyl-trans-4-hydroxy-L- proline |
| 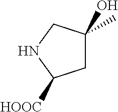 | Cis-4-hydroxy-trans-4-methyl-L- proline |
| 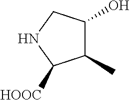 | Cis-3-methyl-trans-4-hydroxy-L- proline |
| 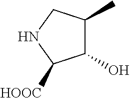 | Cis-4-methyl-trans-2-hydroxy-L- proline |
| 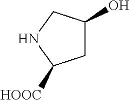 | Cis-4-hydroxy-L-proline |
| 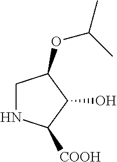 | Cis-4-isopropoxy-trans-2-hydroxy- L-proline |
| 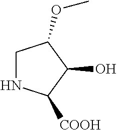 | Trans-4-methoxy-cis-2-hydroxy-L- proline |
| 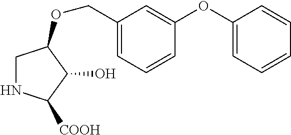 [SMILES: O=C(O)[C@H]1NC[C@@H](OCc2cccc(Oc3ccccc3)c2)[C@@H]1O] | (2R,3S,4S)-3-Hydroxy-4-[(3- phenoxybenzyl)oxy]pyrrolidine-2- carboxylic acid |
| 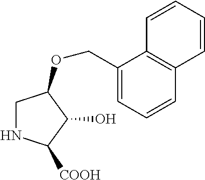 [SMILES: O=C(O)[C@H]1NC[C@@H](OCc2cccc3ccccc23)[C@@H]1O] | (2R,3S,4S)-3-Hydroxy-4- [naphthalene-1- ylmethoxy]pyrrolidine-2-carboxylic acid |
| 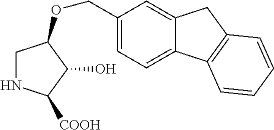 [SMILES: O=C(O)[C@H]1NC[C@@H](OCc2ccc3c(c2)Cc2ccccc2-3)[C@@H]1O] | Fluorenyl |
| 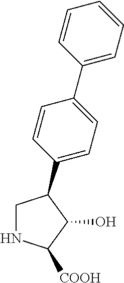 [SMILES: O=C(O)[C@H]1NC[C@@H](c2ccc(-c3ccccc3)cc2)[C@@H]1O] | (2R,3S,4S)-4-([1,1′-Biphenyl]-4-yl)- 3-hydroxypyrrolidine-2-carboxylic acid |
| 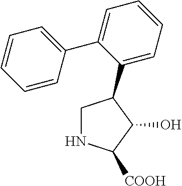 [SMILES: O=C(O)[C@H]1NC[C@@H](c2ccccc2-c2ccccc2)[C@@H]1O] | (2R,3S,4S)-4-([1,1′-Biphenyl]-2-yl)- 3-hydroxypyrrolidine-2-carboxylic acid |
| 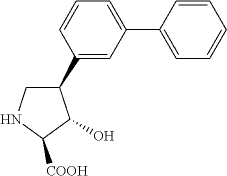 [SMILES: O=C(O)[C@H]1NC[C@@H](c2cccc(-c3ccccc3)c2)[C@@H]1O] | (2R,3S,4S)-4-([1,1′-Biphenyl]-3-yl)- 3-hydroxypyrrolidine-2-carboxylic acid |
| 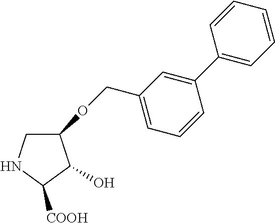 [SMILES: O=C(O)[C@H]1NC[C@@H](OCc2cccc(-c3ccccc3)c2)[C@@H]1O] | (2R,3S,4S)-4-(Biphenyl-3- ylmethoxy)-3-hydroxypyrrolidine-2- carboxylic acid |
| 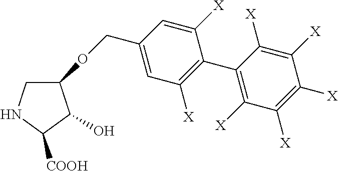 X = H, Cl, F, Br, Me, OMe, iso-Pr, Ph, CF₃ | n/a |

In another aspect, the invention provides a pharmaceutically acceptable salt of a compound selected from:
Trans-3-hydroxy-cis-4-methoxy-L-proline;
cis-3-hydroxy-trans-4-isopropoxy-L-proline;
Trans-3-hydroxy-cis-4-benzyloxy-L-proline;
Trans-3-hydroxy-cis-4-(3-chlorobenzyloxy)-L-proline;
Trans-3-hydroxy-cis-4-(3-fluorobenzyloxy)-L-proline;
Trans-3-hydroxy-cis-4-(3-bromobenzyloxy)-L-proline;
Trans-3-hydroxy-cis-4-(3-methylbenzyloxy)-L-proline;
Trans-3-hydroxy-cis-4-(3-methoxybenzyloxy)-L-proline;
Trans-3-hydroxy-cis-4-(3-isopropyl benzyloxy)-L-proline;
Trans-3-hydroxy-cis-4-(3-phenylbenzyloxy)-L-proline;
Trans-3-hydroxy-cis-4-(3-trifluoromethylbenzyloxy)-L-proline;
Trans-3-hydroxy-cis-4-(4-chlorobenzyloxy)-L-proline;
Trans-3-hydroxy-cis-4-(4-fluorobenzyloxy)-L-proline;
Trans-3-hydroxy-cis-4-(4-bromobenzyloxy)-L-proline;
Trans-3-hydroxy-cis-4-(4-methylbenzyloxy)-L-proline;
Trans-3-hydroxy-cis-4-(4-methoxybenzyloxy)-L-proline;
Trans-3-hydroxy-cis-4-(4-isopropyl benzyloxy)-L-proline;
Trans-3-hydroxy-cis-4-(4-phenylbenzyloxy)-L-proline;
Trans-3-hydroxy-cis-4-(4-trifluoromethylbenzyloxy)-L-proline;
Trans-3-hydroxy-cis-4-(2-chlorobenzyloxy)-L-proline;
Trans-3-hydroxy-cis-4-(2-fluorobenzyloxy)-L-proline;
Trans-3-hydroxy-cis-4-(2-bromobenzyloxy)-L-proline;
Trans-3-hydroxy-cis-4-(2-methylbenzyloxy)-L-proline;
Trans-3-hydroxy-cis-4-(2-methoxybenzyloxy)-L-proline;
Trans-3-hydroxy-cis-4-(2-isopropyl benzyloxy)-L-proline;
Trans-3-hydroxy-cis-4-(2-phenylbenzyloxy)-L-proline;
Trans-3-hydroxy-cis-4-(2-trifluoromethylbenzyloxy)-L-proline;
cis-3-hydroxy-trans-4-benzyloxy-L-proline;
(2R,3S,4S)-4-(biphenyl-4-ylmethoxy)-3-hydroxypyrrolidine-2-carboxylic acid;
(2R,3S,4S)-3-hydroxy-4-((4′-chloro-[1,1′-biphenyl]-4-yl)methoxy)pyrrolidine-2-carboxylic acid;
(2R,3S,4S)-3-hydroxy-4-((4′-fluoro-[1,1′-biphenyl]-4-yl)methoxy)pyrrolidine-2-carboxylic acid;
(2R,3S,4S)-3-hydroxy-4-((4′-bromo-[1,1′-biphenyl]-4-yl)methoxy)pyrrolidine-2-carboxylic acid;
(2R,3S,4S)-3-hydroxy-4-((4′-methyl-[1,1′-biphenyl]-4-yl)methoxy)pyrrolidine-2-carboxylic acid;
(2R,3S,4S)-3-hydroxy-4-((4′-methoxy-[1,1′-biphenyl]-4-yl)methoxy)pyrrolidine-2-carboxylic acid;
(2R,3S,4S)-3-hydroxy-4-((4′-isopropyl-[1,1′-biphenyl]-4-yl)methoxy)pyrrolidine-2-carboxylic acid;
(2R,3S,4S)-3-hydroxy-4-((4′-phenyl-[1,1′-biphenyl]-4-yl)methoxy)pyrrolidine-2-carboxylic acid;
(2R,3S,4S)-3-hydroxy-4-((4′-trifluoromethyl-[1,1′-biphenyl]-4-yl)methoxy)pyrrolidine-2-carboxylic acid;
(2R,3S,4S)-3-hydroxy-4-((3′-chlorol-[1,1′-biphenyl]-4-yl)methoxy)pyrrolidine-2-carboxylic acid;
(2R,3S,4S)-3-hydroxy-4-((3′-fluoro-[1,1′-biphenyl]-4-yl)methoxy)pyrrolidine-2-carboxylic acid;
(2R,3S,4S)-3-hydroxy-4-((3′-bromo-[1,1′-biphenyl]-4-yl)methoxy)pyrrolidine-2-carboxylic acid;
(2R,3S,4S)-3-hydroxy-4-((3′-methyl-[1,1′-biphenyl]-4-yl)methoxy)pyrrolidine-2-carboxylic acid;
(2R,3S,4S)-3-hydroxy-4-((3′-methoxy-[1,1′-biphenyl]-4-yl)methoxy)pyrrolidine-2-carboxylic acid;
(2R,3S,4S)-3-hydroxy-4-((3′-isopropyl-[1,1′-biphenyl]-4-yl)methoxy)pyrrolidine-2-carboxylic acid;
(2R,3S,4S)-3-hydroxy-4-((3′-phenyl-[1,1′-biphenyl]-4-yl)methoxy)pyrrolidine-2-carboxylic acid;
(2R,3S,4S)-3-hydroxy-4-((3′-trifluoromethyl-[1,1′-biphenyl]-4-yl)methoxy)pyrrolidine-2-carboxylic acid;
(2R,3S,4S)-3-hydroxy-4-((2′-chlorol-[1,1′-biphenyl]-4-yl)methoxy)pyrrolidine-2-carboxylic acid;
(2R,3S,4S)-3-hydroxy-4-((2′-fluoro-[1,1′-biphenyl]-4-yl)methoxy)pyrrolidine-2-carboxylic acid;
(2R,3S,4S)-3-hydroxy-4-((2′-bromo-[1,1′-biphenyl]-4-yl)methoxy)pyrrolidine-2-carboxylic acid;
(2R,3S,4S)-3-hydroxy-4-((2′-methyl-[1,1′-biphenyl]-4-yl)methoxy)pyrrolidine-2-carboxylic acid;
(2R,3S,4S)-3-hydroxy-4-((2′-methoxy-[1,1′-biphenyl]-4-yl)methoxy)pyrrolidine-2-carboxylic acid
(2R,3S,4S)-3-hydroxy-4-((2′-isopropyl-[1,1′-biphenyl]-4-yl)methoxy)pyrrolidine-2-carboxylic acid
(2R,3S,4S)-3-hydroxy-4-((2′-phenyl-[1,1′-biphenyl]-4-yl)methoxy)pyrrolidine-2-carboxylic acid
(2R,3S,4S)-3-hydroxy-4-((2′-trifluoromethyl-[1,1′-biphenyl]-4-yl)methoxy)pyrrolidine-2-carboxylic acid;
(2R,3S,4S)-3-hydroxy-4-((2-chlorol-[1,1′-biphenyl]-4-yl)methoxy)pyrrolidine-2-carboxylic acid;
(2R,3S,4S)-3-hydroxy-4-((2-fluoro-[1,1′-biphenyl]-4-yl)methoxy)pyrrolidine-2-carboxylic acid;
(2R,3S,4S)-3-hydroxy-4-((2-bromo-[1,1′-biphenyl]-4-yl)methoxy)pyrrolidine-2-carboxylic acid;
(2R,3S,4S)-3-hydroxy-4-((2-methyl-[1,1′-biphenyl]-4-yl)methoxy)pyrrolidine-2-carboxylic acid;
(2R,3S,4S)-3-hydroxy-4-((2-methoxy-[1,1′-biphenyl]-4-yl)methoxy)pyrrolidine-2-carboxylic acid;
(2R,3S,4S)-3-hydroxy-4-((2-isopropyl-[1,1′-biphenyl]-4-yl)methoxy)pyrrolidine-2-carboxylic acid;
(2R,3S,4S)-3-hydroxy-4-((2-phenyl-[1,1′-biphenyl]-4-yl)methoxy)pyrrolidine-2-carboxylic acid;
(2R,3S,4S)-3-hydroxy-4-((2-trifluoromethyl-[1,1′-biphenyl]-4-yl)methoxy)pyrrolidine-2-carboxylic acid;
(2R,3S,4S)-4-(biphenyl-4-ylmethoxy)-3-hydroxypyrrolidine-2-carboxylic acid;
(2S,3R,4R)-3-hydroxy-4-phenyl-proline;
(2S,3S,4R)-3-phenyl-4-hydroxy-proline;
(2S,3S,4R)-3-hydroxy-4-phenoxy-proline;
(2S,3S,4S)-3-phenoxy-4-hydroxy-proline;
Cis-4-methyl-trans-4-hydroxy-L-proline;
Cis-4-hydroxy-trans-4-methyl-L-proline;
Cis-3-methyl-trans-4-hydroxy-L-proline;
Cis-4-methyl-trans-2-hydroxy-L-proline;
cis-4-hydroxy-L-proline;
cis-4-isopropoxy-trans-2-hydroxy-L-proline;
trans-4-methoxy-cis-2-hydroxy-L-proline;
(2R,3S,4S)-3-hydroxy-4-[(3-phenoxybenzyl)oxy]pyrrolidine-2-carboxylic acid;
(2R,3S,4S)-3-hydroxy-4-[naphthalene-1-ylmethoxy]pyrrolidine-2-carboxylic acid; Fluorenyl;
(2R,3S,4S)-4-([1,1′-biphenyl]-4-yl)-3-hydroxypyrrolidine-2-carboxylic acid;
(2R,3S,4S)-4-([1,1′-biphenyl]-2-yl)-3-hydroxypyrrolidine-2-carboxylic acid;
(2R,3S,4S)-4-([1,1′-biphenyl]-3-yl)-3-hydroxypyrrolidine-2-carboxylic acid; and
(2R,3S,4S)-4-(biphenyl-3-ylmethoxy)-3-hydroxypyrrolidine-2-carboxylic acid.

The term “pharmaceutically acceptable salts” refers to salts or complexes that retain the desired biological activity of the above identified compounds and exhibit minimal or no undesired toxicological effects. The “pharmaceutically acceptable salts” according to the invention include therapeutically active, non-toxic base or acid salt forms, which the compounds of Formula I are able to form.

The acid addition salt form of a compound of Formula I that occurs in its free form as a base can be obtained by treating the free base with an appropriate acid such as an inorganic acid, for example, hydrochloric acid, hydrobromic acid, sulfuric acid, phosphoric acid, nitric acid and the like; or an organic acid such as for example, acetic, hydroxyacetic, propanoic, lactic, pyruvic, malonic, fumaric acid, maleic acid, oxalic acid, tartaric acid, succinic acid, malic acid, ascorbic acid, benzoic acid, tannic acid, pamoic acid, citric, methylsulfonic, ethanesulfonic, benzenesulfonic, formic acid and the like (Handbook of Pharmaceutical Salts, P. Heinrich Stahl& Camille G. Wermuth (Eds), Verlag Helvetica Chimica Acta-Zürich, 2002, 329-345).

The base addition salt form of a compound of Formula I that occurs in its acid form can be obtained by treating the acid with an appropriate base such as an inorganic base, for example, sodium hydroxide, magnesium hydroxide, potassium hydroxide, calcium hydroxide, ammonia and the like; or an organic base such as for example, L-Arginine, ethanolamine, betaine, benzathine, morpholine and the like. (Handbook of Pharmaceutical Salts, P. Heinrich Stahl& Camille G. Wermuth (Eds), Verlag Helvetica Chimica Acta-Zürich, 2002, 329-345).

In another aspect the invention relates to a pharmaceutical composition comprising as active ingredient a therapeutically effective amount of at least one D-serine transporter inhibitor compound as disclosed in US Patent Application Publication No. 2013/0065935 A1 and a pharmaceutically acceptable adjuvant, diluent or carrier.

In another aspect the invention relates to a method for the treatment of visual system disorders caused by a deficit in N-methyl-D-aspartate receptor function, the method comprising administering to a subject in need thereof an ophthalmically acceptable pharmaceutical composition containing a therapeutically effective amount of at least one or more ASCT1 inhibitor compounds and/or at least one or more ASCT2 inhibitor compounds, as disclosed in US Patent Application Publication No. 2013/0065935 A1.

In another aspect the invention relates to a pharmaceutical composition comprising as active ingredient a therapeutically effective amount of at least one or more ASCT1 inhibitor compounds and/or at least one or more ASCT2 inhibitor compounds, as disclosed in US Patent Application Publication No. 2013/0065935 A1, and a pharmaceutically acceptable adjuvant, diluent or carrier, In another aspect the invention relates to a method for the enhancement of visual function, the method comprising administering to a subject in need thereof an ophthalmically acceptable pharmaceutical composition containing a therapeutically effective amount of one or more D-serine transporter inhibitor compounds, as disclosed in US Patent Application Publication No. 2013/0065935 A1.

In another aspect, the present invention relates to a method for the enhancement of visual function comprising administration of one or more D-serine transporter inhibitors by different administration routes. D-serine transporter inhibitors were identified as compounds that inhibit transport mechanisms in neurons and astrocytes, in D-serine transport assays in vitro.

Enhancement of visual function means administering one or more of the D-serine transport inhibitor compounds to improve the visual function, to alleviate its severity, to prevent the onset of a disorder, and to prevent its reoccurrence. Visual function includes visual acuity, visual field, night vision, color vision, dark/light adaptation, contrast sensitivity, binocular vision, motion detection, etc.

In another aspect the present invention relates to a pharmaceutical composition comprising a therapeutically effective amount of at least one D-serine transporter inhibitor compound, said compound being present alone or in combination with one or more pharmaceutically acceptable excipients.

In another aspect the present invention relates to a method for the treatment of visual system disorders caused by a deficit in N-methyl-D-aspartate receptor function, the method comprising administering to a subject in need thereof an ophthalmically acceptable pharmaceutical composition containing a therapeutically effective amount of one or more D-serine transporter inhibitor compounds, as disclosed in US Patent Application Publication No. 2013/0065935 A1.

In another aspect the present invention relates to a pharmaceutical composition comprising a therapeutically effective amount of at least one compound selected from the group consisting of ASCT1 inhibitor compounds, as disclosed in US Patent Application Publication No. 2013/0065935 A1, or at least one compound selected from the group consisting of ASCT2 inhibitor compounds, as disclosed in US Patent Application Publication No. 2013/0065935 A1, or combinations thereof, said compounds being present alone or in combination with one or more pharmaceutically acceptable excipients.

In another aspect the present invention relates to a method for the treatment of visual system disorders caused by a deficit in N-methyl-D-aspartate receptor function, the method comprising administering to a subject in need thereof an ophthalmically acceptable pharmaceutical composition containing a therapeutically effective amount of one or more compounds selected from the group consisting of ASCT1 inhibitor compounds, as disclosed in US Patent Application Publication No. 2013/0065935 A1, or a therapeutically effective amount of one or more compounds selected from the group consisting of ASCT2 inhibitor compounds, as disclosed in US Patent Application Publication No. 2013/0065935 A1, or combinations thereof.

In another aspect the present invention relates to a method for the enhancement of visual function, the method comprising administering to a subject in need thereof an ophthalmically acceptable pharmaceutical composition containing a therapeutically effective amount of one or more compounds selected from the group consisting of ASCT1 inhibitor compounds, as disclosed in US Patent Application Publication No. 2013/0065935 A1, or containing a therapeutically effective amount of one or more compounds selected from the group consisting of ASCT2 inhibitor compounds, as disclosed in US Patent Application Publication No. 2013/0065935 A1, or combinations thereof.

The actual amount of the compound to be administered in any given case will be determined by a physician taking into account the relevant circumstances, such as the severity of the condition, the age and weight of the patient, the patient's general physical condition, the cause of the condition, and the route of administration.

The patient will be administered the compound orally in any acceptable form, such as a tablet, liquid, capsule, powder and the like, or other routes may be desirable or necessary, particularly if the patient suffers from nausea. Such other routes may include, without exception, transdermal, parenteral, subcutaneous, intranasal, via an implant stent, intrathecal, intravitreal, topical to the eye, back of the eye, front of the eye, intramuscular, intravenous, and intrarectal modes of delivery. Additionally, the formulations may be designed to delay release of the active compound over a given period of time, or to carefully control the amount of drug released at a given time during the course of therapy.

In another embodiment of the invention, there are provided pharmaceutical compositions including at least one compound of the invention in a pharmaceutically acceptable carrier thereof. The phrase “pharmaceutically acceptable” means the carrier, diluent or excipient must be compatible with the other ingredients of the formulation and not deleterious to the recipient thereof.

Pharmaceutical compositions of the present invention can be used in the form of a solid, a solution, an emulsion, a dispersion, a patch, a micelle, a liposome, and the like, wherein the resulting composition contains one or more compounds of the present invention, as an active ingredient, in admixture with an organic or inorganic carrier or excipient suitable for enteral or parenteral applications. Invention compounds may be combined, for example, with the usual non-toxic, pharmaceutically acceptable carriers for tablets, pellets, capsules, suppositories, solutions, emulsions, suspensions, and any other form suitable for use. The carriers which can be used include glucose, lactose, gum acacia, gelatin, mannitol, starch paste, magnesium trisilicate, talc, corn starch, keratin, colloidal silica, potato starch, urea, medium chain length triglycerides, dextrans, and other carriers suitable for use in manufacturing preparations, in solid, semisolid, or liquid form. In addition auxiliary, stabilizing, thickening and coloring agents and perfumes may be used. Invention compounds are included in the pharmaceutical composition in an amount sufficient to produce the desired effect upon the process or disease condition.

Pharmaceutical compositions containing invention compounds may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known in the art for the manufacture of pharmaceutical compositions and such compositions may contain one or more agents selected from the group consisting of a sweetening agent such as sucrose, lactose, or saccharin, flavoring agents such as peppermint, oil of wintergreen or cherry, coloring agents and preserving agents in order to provide pharmaceutically elegant and palatable preparations. Tablets containing invention compounds in admixture with non-toxic pharmaceutically acceptable excipients may also be manufactured by known methods. The excipients used may be, for example, (1) inert diluents such as calcium carbonate, lactose, calcium phosphate or sodium phosphate; (2) granulating and disintegrating agents such as corn starch, potato starch or alginic acid; (3) binding agents such as gum tragacanth, corn starch, gelatin or acacia, and (4) lubricating agents such as magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed.

In some cases, formulations for oral use may be in the form of hard gelatin capsules wherein the invention compounds are mixed with an inert solid diluent, for example, calcium carbonate, calcium phosphate or kaolin. They may also be in the form of soft gelatin capsules wherein the invention compounds are mixed with water or an oil medium, for example, peanut oil, liquid paraffin or olive oil.

The pharmaceutical compositions may be in the form of a sterile injectable suspension. This suspension may be formulated according to known methods using suitable dispersing or wetting agents and suspending agents. The sterile injectable preparation may also be a sterile injectable solution or suspension in a non-toxic parenterally-acceptable diluent or solvent, for example, as a solution in 1,3-butanediol. Sterile, fixed oils are conventionally employed as a solvent or suspending medium. For this purpose any bland fixed oil may be employed including synthetic mono- or diglycerides, fatty acids (including oleic acid), naturally occurring vegetable oils like sesame oil, coconut oil, peanut oil, cottonseed oil, etc., or synthetic fatty vehicles like ethyl oleate or the like. Buffers, preservatives, antioxidants, and the like can be incorporated as required.

Pharmaceutical compositions containing invention compounds may be in a form suitable for topical use, for example, as oily suspensions, as solutions or suspensions in aqueous liquids or no aqueous liquids, or as oil-in-water or water-in-oil liquid emulsions. Pharmaceutical compositions may be prepared by combining a therapeutically effective amount of at least one compound according to the present invention, or a pharmaceutically acceptable salt thereof, as an active ingredient with conventional ophthalmically acceptable pharmaceutical excipients and by preparation of unit dosage suitable for topical ocular use. The therapeutically efficient amount typically is between about 0.0001 and about 5% (w/v), preferably about 0.001 to about 2.0% (w/v) in liquid formulations.

For ophthalmic application, preferably solutions are prepared using a physiological saline solution as a major vehicle. The pH of such ophthalmic solutions should preferably be maintained between 4.5 and 8.0 with an appropriate buffer system, a neutral pH being preferred but not essential. The formulations may also contain conventional pharmaceutically acceptable preservatives, stabilizers and surfactants. Preferred preservatives that may be used in the pharmaceutical compositions of the present invention include, but are not limited to, benzalkonium chloride, chlorobutanol, thimerosal, phenylmercuric acetate and phenylmercuric nitrate. A preferred surfactant is, for example, Tween 80. Likewise, various preferred vehicles may be used in the ophthalmic preparations of the present invention. These vehicles include, but are not limited to, polyvinyl alcohol, povidone, hydroxypropyl methyl cellulose, poloxamers, carboxymethyl cellulose, hydroxyethyl cellulose cyclodextrin and purified water.

Tonicity adjustors may be added as needed or convenient. They include, but are not limited to, salts, particularly sodium chloride, potassium chloride, mannitol and glycerin, or any other suitable ophthalmically acceptable tonicity adjustor.

Various buffers and means for adjusting pH may be used so long as the resulting preparation is ophthalmically acceptable. Accordingly, buffers include acetate buffers, citrate buffers, phosphate buffers and borate buffers. Acids or bases may be used to adjust the pH of these formulations as needed.

In a similar manner an ophthalmically acceptable antioxidant for use in the present invention includes, but is not limited to, sodium metabisulfite, sodium thiosulfate, acetylcysteine, butylated hydroxyanisole and butylated hydroxytoluene. Other excipient components which may be included in the ophthalmic preparations are chelating agents. The preferred chelating agent is edentate disodium, although other chelating agents may also be used in place of or in conjunction with it.

The ingredients are usually used in the following amounts:

| Ingredient | Amount (% w/v) |
|---|---|
| active ingredient | about 0.001-5 |
| preservative | 0-0.10 |
| vehicle | 0-40 |
| tonicity adjustor | 0-10 |
| buffer | 0.01-10 |
| pH adjustor | q.s. pH 4.5-7.8 |
| antioxidant | as needed |
| surfactant | as needed |
| purified water | to make 100% |

The actual dose of the active compounds of the present invention depends on the specific compound, and on the condition to be treated; the selection of the appropriate dose is well within the knowledge of the skilled artisan.

The ophthalmic formulations of the present invention are conveniently packaged in forms suitable for metered application, such as in containers equipped with a dropper, to facilitate application to the eye. Containers suitable for drop wise application are usually made of suitable inert, non-toxic plastic material, and generally contain between about 0.5 and about 15 ml solution. One package may contain one or more unit doses. Especially preservative-free solutions are often formulated in non-resalable containers containing up to about ten, preferably up to about five units doses, where a typical unit dose is from one to about 8 drops, preferably one to about 3 drops. The volume of one drop usually is about 20-35 μl (microliter).

Invention compounds may also be administered in the form of suppositories for rectal administration of the drug. These compositions may be prepared by mixing the invention compounds with a suitable non-irritating excipient, such as cocoa butter, synthetic glyceride esters of polyethylene glycols, which are solid at ordinary temperatures, but liquefy and/or dissolve in the rectal cavity to release the drug.

Since individual subjects may present a wide variation in severity of symptoms and each drug has its unique therapeutic characteristics, the precise mode of administration and dosage employed for each subject is left to the discretion of the practitioner.

An opthalmically acceptable pharmaceutical composition is one that can be administered topically to the eye of a subject in need thereof. Comfort to the subject being administered the composition should be maximized, but other considerations, such as drug stability, may necessitate a pharmaceutical composition that provides less than optimal comfort. In such a case, the composition should be formulated such that it is tolerable to a subject being administered the composition topically.

The claimed pharmaceutical composition can be administered topically in the form of solutions or suspensions, ointments, gels, creams, etc. A “pharmaceutically acceptable excipient” is one that is compatible with the active ingredient of the composition and not harmful to the subject being administered the pharmaceutical composition. Solutions for ophthalmic application are often prepared using physiological saline as a major vehicle. Other vehicles include polyvinyl alcohol, povidone, hydroxypropyl methyl cellulose, poloxamers, carboxymethyl cellulose, hydroxyethyl cellulose, and purified water. Examples of useful excipients also include preservatives, buffers, other pH adjustors, tonicity adjustors, surfactants, antioxidants, and chelating agents.

Useful preservatives include benzalkonium chloride, chlorobutanol, thimerosal, phenylmercuric acetate and phenylmercuric nitrate. Examples of buffers include phosphate, borate, sulfate, acetate, and citrate buffers. Acids or bases may be used to adjust the pH of the compositions as needed. Examples of tonicity agents include glycerin, mannitol, sodium chloride and potassium chloride. Useful surfactants include, for example, Tween 80. Examples of ophthalmically acceptable antioxidants include sodium metabisulfite, sodium thiosulfate, acetylcysteine, butylated hydroxyanisole and butylated hydroxytoluene. A useful chelating agent is edentate disodium.

Mixtures of two or more of any suitable excipients may be used.

Aside from topical application to treat diseases affecting the eye including glaucoma, pharmaceutical compositions containing at least one compound of formula (I) can also be administered periocularly, intraocularly, or by other effective means available in the art.

Persons skilled in the art would readily understand that a drug containing one or more of the compounds disclosed herein can be confected as a powder, pill, tablet or the like, or as a solution, emulsion, suspension, aerosol, syrup or elixir suitable for oral or parenteral administration or inhalation. For solid dosage forms or medicaments, non-toxic solid excipients for admixture with compounds disclosed herein include, but are not limited to, pharmaceutical grades of mannitol, lactose, starch, magnesium stearate, sodium saccharin, polyalkylene glycols, talcum, cellulose, glucose, sucrose, and magnesium carbonate. The solid dosage forms may be coated by a material such as glyceryl monostearate or glyceryl distearate, which is utilized in known techniques to delay disintegration and absorption in the gastrointestinal tract for the purpose of providing a sustained action over a longer period. Solid dosage forms may also be coated by the techniques described in U.S. Pat. Nos. 4,256,108, 4,166,452 and 4,265,874 to form osmotic therapeutic tablets for control release.

Pharmaceutically administrable liquid dosage forms can, for example, comprise a solution or suspension of at least one of the compounds disclosed herein and optional pharmaceutical adjutants in a carrier, such as water, saline, aqueous dextrose, glycerol, ethanol and the like. The liquid dosage forms may also contain nontoxic auxiliary substances such as wetting or emulsifying agents, pH buffering agents and the like. Examples of such auxiliary agents include sodium acetate, sorbitan monolaurate, triethanolamine, sodium acetate, triethanolamine oleate, etc. Methods for preparing such dosage forms are well-known to persons skilled in the art (see, for example, Remington's Pharmaceutical Sciences, Mack Publishing Company, Easton, Pa., 16^{th} Edition, 1980).

Parenteral administration is generally characterized by subcutaneous, intramuscular, or intravenous injection. Injectables can be prepared as liquid solutions or suspensions, solid forms that can be reconstituted into solutions or suspensions prior to injection, or as emulsions. Suitable excipients include water, saline dextrose, glycerol, ethanol and the like. Such injectable pharmaceutical compositions may also contain minor amounts of non-toxic auxiliary substances such as wetting or emulsifying agents, pH buffers and the like.

The following examples are for illustrative purposes only and are not intended, nor should they be construed as limiting the invention in any manner. Those skilled in the art will appreciate that variations and modifications of the following examples can be made without exceeding the spirit or scope of the invention.

### EXAMPLES

### General Procedures Followed in Obtaining Experimental Data Transport Experiments

Cell-based assays: the transport of [³H]L- or D-serine was measured in primary cultures of rat hippocampal astrocytes or in human embryonic kidney (HEK) cells expressing ASCT transporter sub-types. For the astrocyte assays, cells were plated on either 24- or 96-well plates at a density of 50,000 cells per well. For the HEK assays, cells were plated on coated 96-well plates at a density of 80,000 cells/well. Assays were conducted in duplicate at room temperature in assay buffer consisting of: NaCl: 150 mM, KCl: 2 mM; MgCl₂: 1 mM; CaCl₂: 1 mM; HEPES: Tris buffer: 10 mM, pH7.4. To assess the sodium-dependence of transport, NaCl was replaced in the assay buffer by equimolar choline chloride. Following aspiration of growth medium and 2 washes with assay buffer, cells were incubated with [³H]L- or D-serine at a final concentration of 1 μM for 5 min (astrocytes) or 1 min (HEK cells), after which the incubation medium was aspirated and the cells washed twice with ice-cold assay buffer. Cells containing radiolabel were solubilized in 100 μl of 1% Triton-X100 and an aliquot counted in a beta counter. IC₅₀ values were determined over a range of at least 6 concentrations and derived from curve-fitting algorithms available in GraphPad Prism 4. Table 2 reflects the IC₅₀ values for the inhibition of [³H]L-serine transport into astrocytes and HEK cells expressing recombinant human ASCT1 or ASCT2. Values are IC₅₀'s in μM from 6-12 point inhibition curves, with an n of 3-4.

TABLE 2

| | Astro | HEK1A4 | HEK1A5 |
|---|---|---|---|
| | ASCT1/2 | ASCT1 | ASCT2 |
| Compound | IC₅₀ μM | IC₅₀ μM | IC₅₀ μM |
| (2R,3S,4S)-4-(biphenyl-4- | | | |
| ylmethoxy)-3-hydroxypyrrolidine- | 0.60 | 3.6 | 2.8 |
| 2-carboxylic acid | | | |
| (2R,3S,4S)-3-hydroxy-4-[(3- | | | |
| phenoxybenzyl)oxy]pyrrolidine-2- | 10.7 | 46.4 | 65.2 |
| carboxylic acid | | | |

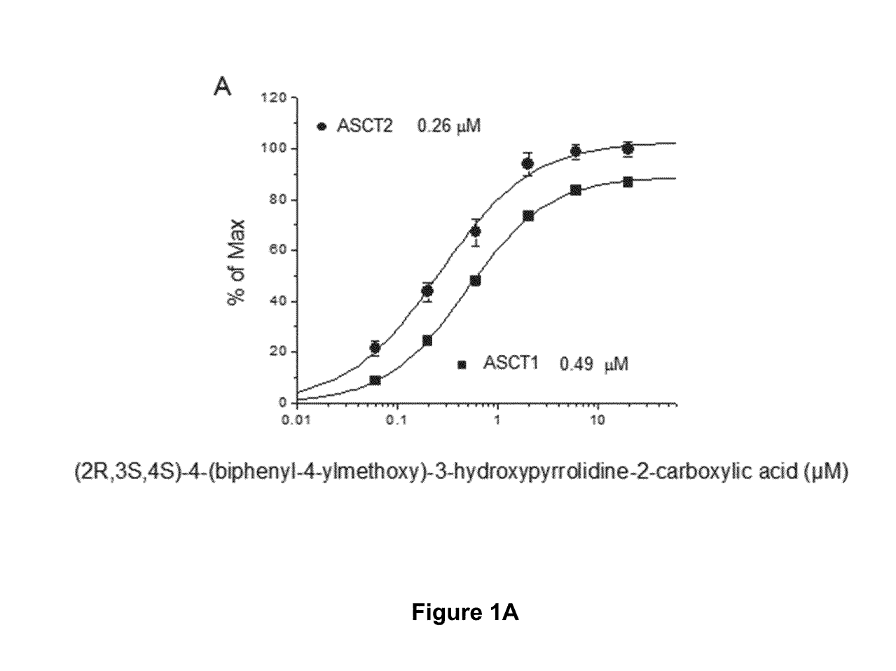

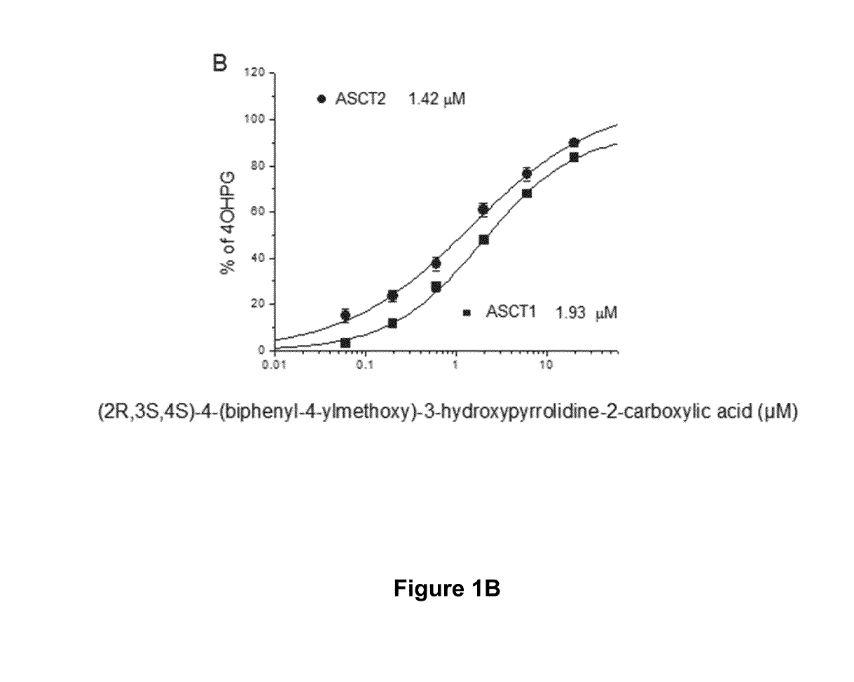

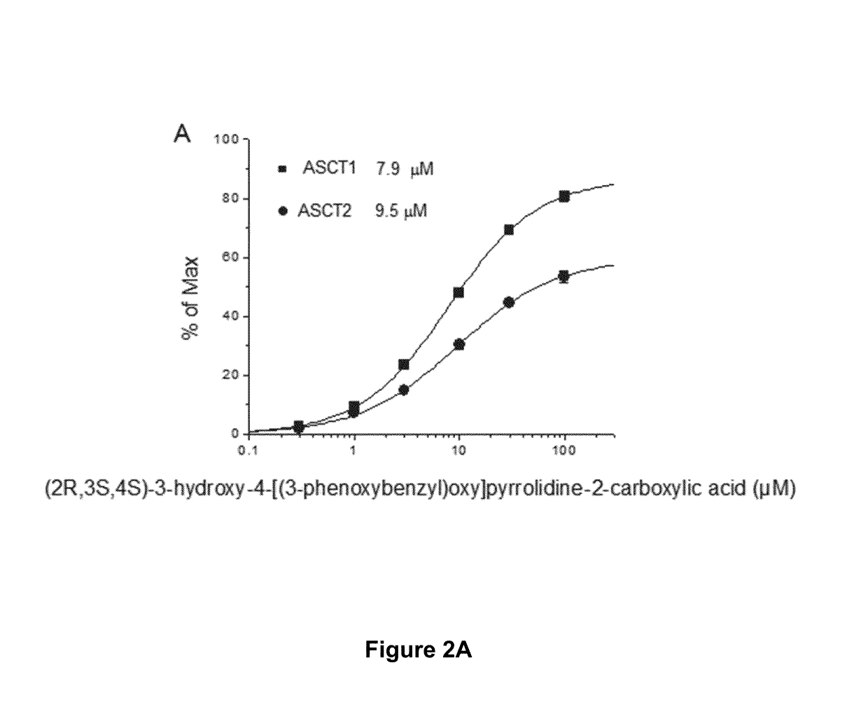

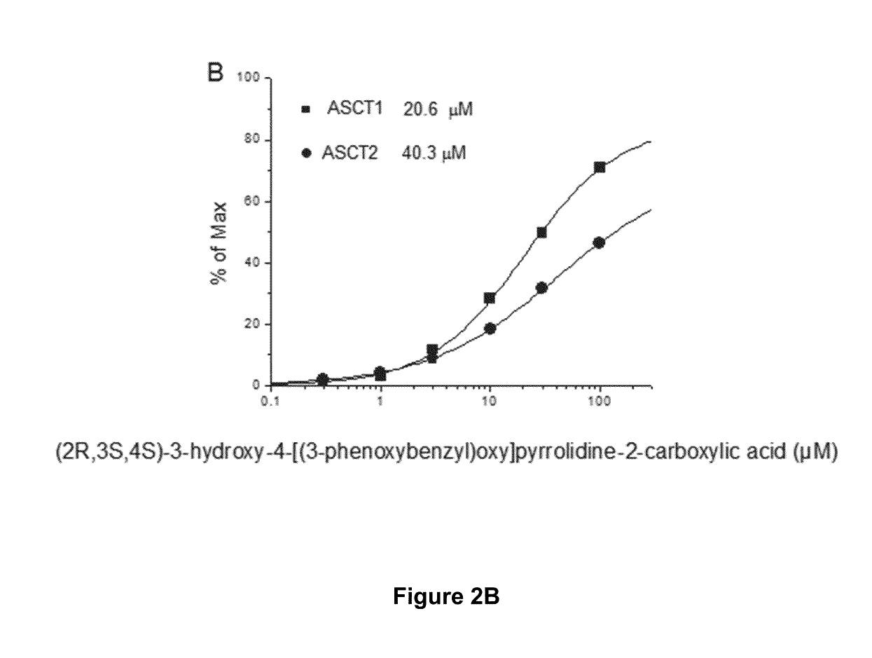

Patch-Clamp Recording in HEK 293 Cells (FIGS. 1A, 1B, 2A and 2B):

Transport currents were recorded in human embryonic kidney (HEK) cells expressing human ASCT1 or ASCT2 transporter using whole cell patch-clamp. Extracellular solution contains: 140 mM NaCl, 2.0 mM MgCl₂, 2.0 CaCl₂, 10 mM HEPES, pH 7.4. The recording pipettes were filled with ICM containing: 130 mM NaSCN, 10 mM EGTA, 2 mM MgCl₂, 10 mM HEPES, 10 mM L-alanine pH 7.3. The inhibitory effects of compounds were measured without/with 300 μM L-serine and normalized to these by 4-hydroxy-phenyglycine (100%).

FIGS. 1A, 1B, 2A and 2B shown the dose-dependent effects of (2R,3S,4S)-4-(biphenyl-4-ylmethoxy)-3-hydroxypyrrolidine-2-carboxylic acid and (2R,3S,4S)-3-hydroxy-4-[(3-phenoxybenzyl)oxy]pyrrolidine-2-carboxylic acid on transport currents with or without substrate L-serine. The IC₅₀ values estimated based on the transport current agree well with those in Table 2 from transport experiments using radiolabelled substrate.

### Visual Cortex Slice Physiology:

Following decapitation of the rat, the brain was rapidly removed and immersed in ice-cold artificial cerebrospinal fluid (ACSF) containing 124 mM NaCl, 3 mM KCl, 1.25 mM KH₂PO₄, 3.4 mM CaCl₂, 2.5 mM MgSO₄, 26 mM NaHCO₃, and 10 mM D-glucose. A block of visual cortex was created by removing the frontal ⅔ portion of the brain and the cerebellum. Coronal visual cortex slices of 375 μm were prepared from adult Sprague Dawley (SD) rats using a vibratome (VT 1000 S; Leica). The slices were maintained in an interface recording chamber perfused with preheated ACSF. Slices were continuously perfused with this solution at a rate of 1.00-1.50 ml/min while the surface of the slices was exposed to warm, humidified 95% O₂/5% CO₂ and maintained at 31±1° C. Visual cortex slices were allowed to recover for 1 hr before recording began. A single stimulating and recording electrode were placed in layer IV and III, respectively, to generate and record a field excitatory postsynaptic potentials (fEPSPs). Pulses were administered every 20 s using a current that produced a fEPSP that was 50% of the maximum spike free response. An input-output (IO) curve was done to determine the stimulation needed to achieve a stable baseline. Following a 15 min stable baseline recording period, a train of 5 theta bursts (each burst containing four pulses at 100 Hz with an inter-burst interval of 200 ms) were delivered to the slice. This was repeated 2 additional times with a 1 minute intertrain interval, and the level of LTP was recorded for at least 30 min. Changes in amplitude of the synaptic response were used to measure the extent of LTP because it was determined to be the more consistent parameter than the slope of the response. Control LTP values were obtained from slices not treated with drug. Different slices were used to study drug effects on LTP. After a 15 min baseline recording period, the compounds of interest were infused for 15 minutes followed by LTP induction. Washout of the compounds began 5 minutes after tetanization. Recording of the amplitude before, during, and after drug infusion was done.

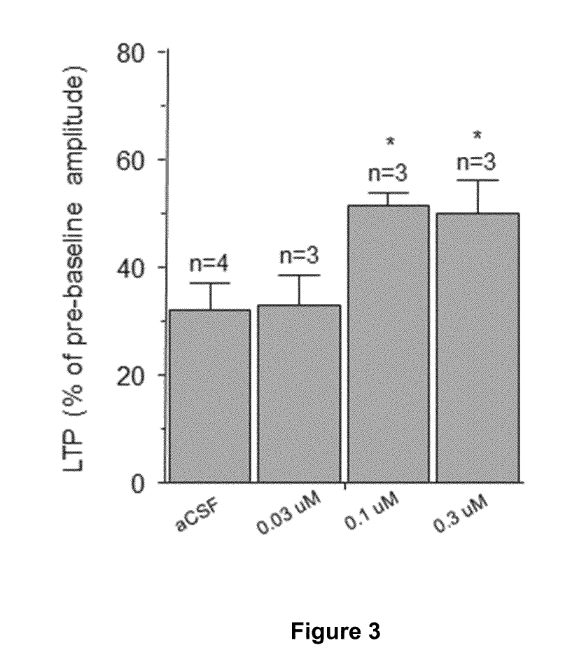

Long Term Potentiation (LTP) in primary visual cortex has been used as a cellular model for visual cortex plasticity and has functional consequences on visual evoked responses. NMDA receptors play a critical role in visual cortex LTP induction. FIG. 3 showed that (2R,3S,4S)-4-(biphenyl-4-ylmethoxy)-3-hydroxypyrrolidine-2-carboxylic acid enhanced LTP in visual cortex at concentration as low as 0.1 μM.

Two proline analogs were evaluated for their ability to inhibit the neutral amino acid transporters ASCT1 and ASCT2 for which D-serine is a substrate. In rat brain astrocytes in culture, which endogenously express both ASCT1 and ASCT2, Compounds (2R,3S,4S)-4-(biphenyl-4-ylmethoxy)-3-hydroxypyrrolidine-2-carboxylic acid and (2R,3S,4S)-3-hydroxy-4-[(3-phenoxybenzyl)oxy]pyrrolidine-2-carboxylic acid both completely inhibited radiolabelled substrate transport with IC₅₀ values of 0.6 and 10.7 (Table 2). In human embryonic kidney (HEK) cells with heterologous expression of human ASCT1 or ASCT2, both compounds inhibited radiolabelled substrate transport with IC₅₀ values that were similar in both cell lines indicating that these compounds inhibit both human transporter sub-types (Table 2). Patch clamp studies also showed that (2R,3S,4S)-4-(biphenyl-4-ylmethoxy)-3-hydroxypyrrolidine-2-carboxylic acid and (2R,3S,4S)-3-hydroxy-4-[(3-phenoxybenzyl)oxy]pyrrolidine-2-carboxylic acid inhibit human ASCT1 and ASCT2 expressed in HEK cells. Both compounds evoked an outward current in the absence of applied substrate (FIGS. 1 and 2) that is typical of compounds that act as transport inhibitors. In the presence of substrate (0.3 mM L-serine), both compounds inhibited the substrate-evoked inward current in both cell lines (FIGS. 1 and 2). The IC₅₀ values (in presence of substrate) from the patch clamp experiments with human ASCT1 and ASCT2 (FIGS. 1 and 2) agree well with the IC₅₀ values obtained in these cell lines from the radiolabelled transport studies (Table 2). Consequently, these experiments show that (2R,3S,4S)-4-(biphenyl-4-ylmethoxy)-3-hydroxypyrrolidine-2-carboxylic acid and (2R,3S,4S)-3-hydroxy-4-[(3-phenoxybenzyl)oxy]pyrrolidine-2-carboxylic acid are effective inhibitors of ASCT1 and ASCT2 in both native and heterologous expression systems using both patch clamp and radiolabelled substrate assays. We have previously shown in US Patent Publication Application No. 2012/0302621, that inhibitors of ASCT1 and ASCT2 can potentiate NMDA-receptor-mediated synaptic responses through elevation of extracellular D-serine. Therefore we showed that (2R,3S,4S)-4-(biphenyl-4-ylmethoxy)-3-hydroxypyrrolidine-2-carboxylic acid have the ability to enhance NMDA-receptor-mediated synaptic responses such as long-term potentiation (LTP). In the rat visual cortex slice, (2R,3S,4S)-4-(biphenyl-4-ylmethoxy)-3-hydroxypyrrolidine-2-carboxylic acid potently enhanced (LTP) with significant effects at 0.1 and 0.3 μM (FIG. 3). Overall, these findings indicate that proline analogs such as (2R,3S,4S)-4-(biphenyl-4-ylmethoxy)-3-hydroxypyrrolidine-2-carboxylic acid and (2R,3S,4S)-3-hydroxy-4-[(3-phenoxybenzyl)oxy]pyrrolidine-2-carboxylic acid enhance visual function and have the potential to improve visual function when it has been impaired by disease.

Various modifications of the present invention, in addition to those shown and described herein, will be apparent to those skilled in the art of the above description. Such modifications are also intended to fall within the scope of the appended claims.

Patents and publications mentioned in the specification are indicative of the levels of those skilled in the art to which the invention pertains. These patents and publications are incorporated herein by reference to the same extent as if each individual application or publication was specifically and individually incorporated herein by reference.

The foregoing description is illustrative of particular embodiments of the invention, but is not meant to be a limitation upon the practice thereof. The following claims, including all equivalents thereof, are intended to define the scope of the invention.

We claim:

1. A method for the treatment of visual system disorders caused by a deficit in N-methyl-D-aspartate receptor function, the method comprising administering to a subject in need thereof an ophthalmically acceptable pharmaceutical composition containing a therapeutically effective amount of one or more D-serine transporter inhibitor compounds which are proline analogues.

2. The method according to claim 1, wherein the visual system disorders are selected from: macular edema, dry and wet macular degeneration, choroidal neovascularization, diabetic retinopathy, acute macular neuroretinopathy, central serous chorioretinopathy, cystoid macular edema, and diabetic macular edema, uveitis, retinitis, choroiditis, acute multifocal placoid pigment epitheliopathy, Behcet's disease, birdshot retinochoroidopathy, syphilis, lyme, tuberculosis, toxoplasmosis, intermediate uveitis (pars planitis), multifocal choroiditis, multiple evanescent white dot syndrome (mewds), ocular sarcoidosis, posterior scleritis, serpiginous choroiditis, subretinal fibrosis and uveitis syndrome, Vogt-Koyanagi—and Harada syndrome; retinal arterial occlusive disease, anterior uveitis, retinal vein occlusion, central retinal vein occlusion, disseminated intravascular coagulopathy, branch retinal vein occlusion, hypertensive fundus changes, ocular ischemic syndrome, retinal arterial microaneurysms, Coat's disease, parafoveal telangiectasis, hemiretinal vein occlusion, papillophlebitis, central retinal artery occlusion, branch retinal artery occlusion, carotid artery disease (CAD), frosted branch angiitis, sickle cell retinopathy, angioid streaks, familial exudative vitreoretinopathy, and Eales disease; traumatic/surgical conditions such as sympathetic ophthalmia, uveitic retinal disease, retinal detachment, trauma, photocoagulation, hypoperfusion during surgery, radiation retinopathy, and bone marrow transplant retinopathy; proliferative vitreal retinopathy and epiretinal membranes, and proliferative diabetic retinopathy; infectious disorders such as ocular histoplasmosis, ocular toxocariasis, presumed ocular histoplasmosis syndrome (PONS), endophthalmitis, toxoplasmosis, retinal diseases associated with HIV infection, choroidal disease associate with HIV infection, uveitic disease associate with HIV infection, viral retinitis, acute retinal necrosis, progressive outer retinal necrosis, fungal retinal diseases, ocular syphilis, ocular tuberculosis, diffuse unilateral subacute neuroretinitis, and myiasis; genetic disorders such as retinitis pigmentosa, systemic disorders with associated retinal dystrophies, congenital stationary night blindness, cone dystrophies, Stargardt's disease and fundus flavimaculatus, Best's disease, pattern dystrophy of the retinal pigmented epithelium, X-linked retinoschisis, Sorsby's fundus dystrophy, benign concentric maculopathy, Bietti's crystalline dystrophy, and pseudoxanthoma elasticum; retinal tears/holes such as retinal detachment, macular hole, and giant retinal tear; tumors such as retinal disease associated with tumors, congenital hypertrophy of the retinal pigmented epithelium, posterior uveal melanoma, choroidal hemangioma, choroidal osteoma, choroidal metastasis, combined hamartoma of the retina and retinal pigmented epithelium, retinoblastoma, vasoproliferative tumors of the ocular fundus, retinal astrocytoma, and intraocular lymphoid tumors; punctate inner choroidopathy, acute posterior multifocal placoid pigment epitheliopathy, myopic retinal degeneration, acute retinal pigement epitheliitis, retinitis pigmentosa, proliferative vitreal retinopathy (PVR), age-related macular degeneration (ARMD), diabetic retinopathy, diabetic macular edema, retinal detachment, retinal tear, uveitus, cytomegalovirus retinitis, glaucoma, amblyopia, stroke-induced blindness, visual dysfunction in Parkinson's disease, Alzheimer's disease and multiple sclerosis, seizure-induced cortical blindness, induced visual dysfunction and epileptic blindness.

3. The method according to claim 1, wherein the proline analogue is selected from:
Trans-3-hydroxy-cis-4-methoxy-L-proline;
cis-3-hydroxy-trans-4-isopropoxy-L-proline;
Trans-3-hydroxy-cis-4-benzyloxy-L-proline;
Trans-3-hydroxy-cis-4-(3-chlorobenzyloxy)-L-proline;
Trans-3-hydroxy-cis-4-(3-fluorobenzyloxy)-L-proline;
Trans-3-hydroxy-cis-4-(3-bromobenzyloxy)-L-proline;
Trans-3-hydroxy-cis-4-(3-methylbenzyloxy)-L-proline;
Trans-3-hydroxy-cis-4-(3-methoxybenzyloxy)-L-proline;
Trans-3-hydroxy-cis-4-(3-isopropylbenzyloxy)-L-proline;
Trans-3-hydroxy-cis-4-(3-phenylbenzyloxy)-L-proline;
Trans-3-hydroxy-cis-4-(3-trifluoromethylbenzyloxy)-L-proline;
Trans-3-hydroxy-cis-4-(4-chlorobenzyloxy)-L-proline;
Trans-3-hydroxy-cis-4-(4-fluorobenzyloxy)-L-proline;
Trans-3-hydroxy-cis-4-(4-bromobenzyloxy)-L-proline;
Trans-3-hydroxy-cis-4-(4-methylbenzyloxy)-L-proline;
Trans-3-hydroxy-cis-4-(4-methoxybenzyloxy)-L-proline;
Trans-3-hydroxy-cis-4-(4-isopropylbenzyloxy)-L-proline;
Trans-3-hydroxy-cis-4-(4-phenylbenzyloxy)-L-proline;
Trans-3-hydroxy-cis-4-(4-trifluoromethylbenzyloxy)-L-proline;
Trans-3-hydroxy-cis-4-(2-chlorobenzyloxy)-L-proline;
Trans-3-hydroxy-cis-4-(2-fluorobenzyloxy)-L-proline;
Trans-3-hydroxy-cis-4-(2-bromobenzyloxy)-L-proline;
Trans-3-hydroxy-cis-4-(2-methylbenzyloxy)-L-proline;
Trans-3-hydroxy-cis-4-(2-methoxybenzyloxy)-L-proline;
Trans-3-hydroxy-cis-4-(2-isopropylbenzyloxy)-L-proline;
Trans-3-hydroxy-cis-4-(2-phenylbenzyloxy)-L-proline;
Trans-3-hydroxy-cis-4-(2-trifluoromethylbenzyloxy)-L-proline;
cis-3-hydroxy-trans-4-benzyloxy-L-proline;
(2R,3S,4S)-4-(biphenyl-4-ylmethoxy)-3-hydroxypyrrolidine-2-carboxylic acid;
(2R,3S,4S)-3-hydroxy-4-((4′-chloro-[1,1′-biphenyl]-4-yl)methoxy)pyrrolidine-2-carboxylic acid;
(2R,3S,4S)-3-hydroxy-4-((4′-fluoro-[1,1′-biphenyl]-4-yl)methoxy)pyrrolidine-2-carboxylic acid;
(2R,3S,4S)-3-hydroxy-4-((4′-bromo-[1,1′-biphenyl]-4-yl)methoxy)pyrrolidine-2-carboxylic acid;
(2R,3S,4S)-3-hydroxy-4-((4′-methyl-[1,1′-biphenyl]-4-yl)methoxy)pyrrolidine-2-carboxylic acid;
(2R,3S,4S)-3-hydroxy-4-((4′-methoxy-[1,1′-biphenyl]-4-yl)methoxy)pyrrolidine-2-carboxylic acid;
(2R,3S,4S)-3-hydroxy-4-((4′-isopropyl-[1,1′-biphenyl]-4-yl)methoxy)pyrrolidine-2-carboxylic acid;
(2R,3S,4S)-3-hydroxy-4-((4′-phenyl-[1,1′-biphenyl]-4-yl)methoxy)pyrrolidine-2-carboxylic acid;
(2R,3S,4S)-3-hydroxy-4-((4′-trifluoromethyl-[1,1′-biphenyl]-4-yl)methoxy)pyrrolidine-2-carboxylic acid;
(2R,3S,4S)-3-hydroxy-4-((3′-chlorol-[1,1′-biphenyl]-4-yl)methoxy)pyrrolidine-2-carboxylic acid;
(2R,3S,4S)-3-hydroxy-4-((3′-fluoro-[1,1′-biphenyl]-4-yl)methoxy)pyrrolidine-2-carboxylic acid;
(2R,3S,4S)-3-hydroxy-4-((3′-bromo-[1,1′-biphenyl]-4-yl)methoxy)pyrrolidine-2-carboxylic acid;
(2R,3S,4S)-3-hydroxy-4-((3′-methyl-[1,1′-biphenyl]-4-yl)methoxy)pyrrolidine-2-carboxylic acid;
(2R,3S,4S)-3-hydroxy-4-((3′-methoxy-[1,1′-biphenyl]-4-yl)methoxy)pyrrolidine-2-carboxylic acid;
(2R,3S,4S)-3-hydroxy-4-((3′-isopropyl-[1,1′-biphenyl]-4-yl)methoxy)pyrrolidine-2-carboxylic acid;
(2R,3S,4S)-3-hydroxy-4-((3′-phenyl-[1,1′-biphenyl]-4-yl)methoxy)pyrrolidine-2-carboxylic acid;
(2R,3S,4S)-3-hydroxy-4-((3′-trifluoromethyl-[1,1′-biphenyl]-4-yl)methoxy)pyrrolidine-2-carboxylic acid;
(2R,3S,4S)-3-hydroxy-4-((2′-chlorol-[1,1′-biphenyl]-4-yl)methoxy)pyrrolidine-2-carboxylic acid;
(2R,3S,4S)-3-hydroxy-4-((2′-fluoro-[1,1′-biphenyl]-4-yl)methoxy)pyrrolidine-2-carboxylic acid;
(2R,3S,4S)-3-hydroxy-4-((2′-bromo-[1,1′-biphenyl]-4-yl)methoxy)pyrrolidine-2-carboxylic acid;
(2R,3S,4S)-3-hydroxy-4-((2′-methyl-[1,1′-biphenyl]-4-yl)methoxy)pyrrolidine-2-carboxylic acid;
(2R,3S,4S)-3-hydroxy-4-((2′-methoxy-[1,1′-biphenyl]-4-yl)methoxy)pyrrolidine-2-carboxylic acid
(2R,3S,4S)-3-hydroxy-4-((2′-isopropyl-[1,1′-biphenyl]-4-yl)methoxy)pyrrolidine-2-carboxylic acid
(2R,3S,4S)-3-hydroxy-4-((2′-phenyl-[1,1′-biphenyl]-4-yl)methoxy)pyrrolidine-2-carboxylic acid
(2R,3S,4S)-3-hydroxy-4-((2′-trifluoromethyl-[1,1′-biphenyl]-4-yl)methoxy)pyrrolidine-2-carboxylic acid;
(2R,3S,4S)-3-hydroxy-4-((2-chlorol-[1,1′-biphenyl]-4-yl)methoxy)pyrrolidine-2-carboxylic acid;
(2R,3S,4S)-3-hydroxy-4-((2-fluoro-[1,1′-biphenyl]-4-yl)methoxy)pyrrolidine-2-carboxylic acid;
(2R,3S,4S)-3-hydroxy-4-((2-bromo-[1,1′-biphenyl]-4-yl)methoxy)pyrrolidine-2-carboxylic acid;
(2R,3S,4S)-3-hydroxy-4-((2-methyl-[1,1′-biphenyl]-4-yl)methoxy)pyrrolidine-2-carboxylic acid;
(2R,3S,4S)-3-hydroxy-4-((2-methoxy-[1,1′-biphenyl]-4-yl)methoxy)pyrrolidine-2-carboxylic acid;
(2R,3S,4S)-3-hydroxy-4-((2-isopropyl-[1,1′-biphenyl]-4-yl)methoxy)pyrrolidine-2-carboxylic acid;
(2R,3S,4S)-3-hydroxy-4-((2-phenyl-[1,1′-biphenyl]-4-yl)methoxy)pyrrolidine-2-carboxylic acid;
(2R,3S,4S)-3-hydroxy-4-((2-trifluoromethyl-[1,1′-biphenyl]-4-yl)methoxy)pyrrolidine-2-carboxylic acid;
(2R,3S,4S)-4-(biphenyl-4-ylmethoxy)-3-hydroxypyrrolidine-2-carboxylic acid;
(2S,3R,4R)-3-hydroxy-4-phenyl-proline;
(2S,3S,4R)-3-phenyl-4-hydroxy-proline;
(2S,3S,4R)-3-hydroxy-4-phenoxy-proline;
(2S,3S,4S)-3-phenoxy-4-hydroxy-proline;
Cis-4-methyl-trans-4-hydroxy-L-proline;
Cis-4-hydroxy-trans-4-methyl-L-proline;
Cis-3-methyl-trans-4-hydroxy-L-proline;
Cis-4-methyl-trans-2-hydroxy-L-proline;
cis-4-hydroxy-L-proline;
cis-4-isopropoxy-trans-2-hydroxy-L-proline;
trans-4-methoxy-cis-2-hydroxy-L-proline;
(2R,3S,4S)-3-hydroxy-4-[(3-phenoxybenzyl)oxy]pyrrolidine-2-carboxylic acid;
(2R,3S,4S)-3-hydroxy-4-[naphthalene-1-ylmethoxy]pyrrolidine-2-carboxylic acid;
Fluorenyl;
(2R,3S,4S)-4-([1,1′-biphenyl]-4-yl)-3-hydroxypyrrolidine-2-carboxylic acid;
(2R,3S,4S)-4-([1,1′-biphenyl]-2-yl)-3-hydroxypyrrolidine-2-carboxylic acid;
(2R,3S,4S)-4-([1 i′-biphenyl]-3-yl)-3-hydroxypyrrolidine-2-carboxylic acid; and
(2R,3S,4S)-4-(biphenyl-3-ylmethoxy)-3-hydroxypyrrolidine-2-carboxylic acid,
or a pharmaceutically acceptable salt thereof.

4. The method according to claim 1, wherein the pharmaceutical composition further comprises a pharmaceutically acceptable adjuvant, diluent or carrier.

5. A method for the enhancement of the visual function, the method comprising administering to a subject in need thereof, an ophthalmically acceptable pharmaceutical composition containing a therapeutically effective amount of one or more D-serine transporter inhibitor compounds which are proline analogues.

6. The method according to claim 5, wherein the proline analogue is selected from:
Trans-3-hydroxy-cis-4-methoxy-L-proline;
cis-3-hydroxy-trans-4-isopropoxy-L-proline;
Trans-3-hydroxy-cis-4-benzyloxy-L-proline;
Trans-3-hydroxy-cis-4-(3-chlorobenzyloxy)-L-proline;
Trans-3-hydroxy-cis-4-(3-fluorobenzyloxy)-L-proline;
Trans-3-hydroxy-cis-4-(3-bromobenzyloxy)-L-proline;
Trans-3-hydroxy-cis-4-(3-methylbenzyloxy)-L-proline;
Trans-3-hydroxy-cis-4-(3-methoxybenzyloxy)-L-proline;
Trans-3-hydroxy-cis-4-(3-isopropylbenzyloxy)-L-proline;
Trans-3-hydroxy-cis-4-(3-phenylbenzyloxy)-L-proline;
Trans-3-hydroxy-cis-4-(3-trifluoromethylbenzyloxy)-L-proline;
Trans-3-hydroxy-cis-4-(4-chlorobenzyloxy)-L-proline;
Trans-3-hydroxy-cis-4-(4-fluorobenzyloxy)-L-proline;
Trans-3-hydroxy-cis-4-(4-bromobenzyloxy)-L-proline;
Trans-3-hydroxy-cis-4-(4-methylbenzyloxy)-L-proline;
Trans-3-hydroxy-cis-4-(4-methoxybenzyloxy)-L-proline;
Trans-3-hydroxy-cis-4-(4-isopropylbenzyloxy)-L-proline;
Trans-3-hydroxy-cis-4-(4-phenylbenzyloxy)-L-proline;
Trans-3-hydroxy-cis-4-(4-trifluoromethylbenzyloxy)-L-proline;
Trans-3-hydroxy-cis-4-(2-chlorobenzyloxy)-L-proline;
Trans-3-hydroxy-cis-4-(2-fluorobenzyloxy)-L-proline;
Trans-3-hydroxy-cis-4-(2-bromobenzyloxy)-L-proline;
Trans-3-hydroxy-cis-4-(2-methylbenzyloxy)-L-proline;
Trans-3-hydroxy-cis-4-(2-methoxybenzyloxy)-L-proline;
Trans-3-hydroxy-cis-4-(2-isopropylbenzyloxy)-L-proline;
Trans-3-hydroxy-cis-4-(2-phenylbenzyloxy)-L-proline;
Trans-3-hydroxy-cis-4-(2-trifluoromethylbenzyloxy)-L-proline;
cis-3-hydroxy-trans-4-benzyloxy-L-proline;
(2R,3S,4S)-4-(biphenyl-4-ylmethoxy)-3-hydroxypyrrolidine-2-carboxylic acid;
(2R,3S,4S)-3-hydroxy-4-((4′-chlorol-[1,1′-biphenyl]-4-yl)methoxy)pyrrolidine-2-carboxylic acid;
(2R,3S,4S)-3-hydroxy-4-((4′-fluoro-[1,1′-biphenyl]-4-yl)methoxy)pyrrolidine-2-carboxylic acid;
(2R,3S,4S)-3-hydroxy-4-((4′-bromo-[1,1′-biphenyl]-4-yl)methoxy)pyrrolidine-2-carboxylic acid;
(2R,3S,4S)-3-hydroxy-4-((4′-methyl-[1,1′-biphenyl]-4-yl)methoxy)pyrrolidine-2-carboxylic acid;
(2R,3S,4S)-3-hydroxy-4-((4′-methoxy-[1,1′-biphenyl]-4-yl)methoxy)pyrrolidine-2-carboxylic acid;
(2R,3S,4S)-3-hydroxy-4-((4′-isopropyl-[1,1′-biphenyl]-4-yl)methoxy)pyrrolidine-2-carboxylic acid;
(2R,3S,4S)-3-hydroxy-4-((4′-phenyl-[1,1′-biphenyl]-4-yl)methoxy)pyrrolidine-2-carboxylic acid;
(2R,3S,4S)-3-hydroxy-4-((4′-trifluoromethyl-[1,1′-biphenyl]-4-yl)methoxy)pyrrolidine-2-carboxylic acid;
(2R,3S,4S)-3-hydroxy-4-((3′-chlorol-[1,1′-biphenyl]-4-yl)methoxy)pyrrolidine-2-carboxylic acid;
(2R,3S,4S)-3-hydroxy-4-((3′-fluoro-[1,1′-biphenyl]-4-yl)methoxy)pyrrolidine-2-carboxylic acid;
(2R,3S,4S)-3-hydroxy-4-((3′-bromo-[1,1′-biphenyl]-4-yl)methoxy)pyrrolidine-2-carboxylic acid;
(2R,3S,4S)-3-hydroxy-4-((3′-methyl-[1,1′-biphenyl]-4-yl)methoxy)pyrrolidine-2-carboxylic acid;
(2R,3S,4S)-3-hydroxy-4-((3′-methoxy-[1,1′-biphenyl]-4-yl)methoxy)pyrrolidine-2-carboxylic acid;
(2R,3S,4S)-3-hydroxy-4-((3′-isopropyl-[1,1′-biphenyl]-4-yl)methoxy)pyrrolidine-2-carboxylic acid;
(2R,3S,4S)-3-hydroxy-4-((3′-phenyl-[1,1′-biphenyl]-4-yl)methoxy)pyrrolidine-2-carboxylic acid;
(2R,3S,4S)-3-hydroxy-4-((3′-trifluoromethyl-[1,1′-biphenyl]-4-yl)methoxy)pyrrolidine-2-carboxylic acid;
(2R,3S,4S)-3-hydroxy-4-((2′-chlorol-[1,1′-biphenyl]-4-yl)methoxy)pyrrolidine-2-carboxylic acid;
(2R,3S,4S)-3-hydroxy-4-((2′-fluoro-[1,1′-biphenyl]-4-yl)methoxy)pyrrolidine-2-carboxylic acid;
(2R,3S,4S)-3-hydroxy-4-((2′-bromo-[1,1′-biphenyl]-4-yl)methoxy)pyrrolidine-2-carboxylic acid;
(2R,3S,4S)-3-hydroxy-4-((2′-methyl-[1,1′-biphenyl]-4-yl)methoxy)pyrrolidine-2-carboxylic acid;
(2R,3S,4S)-3-hydroxy-4-((2′-methoxy-[1,1′-biphenyl]-4-yl)methoxy)pyrrolidine-2-carboxylic acid
(2R,3S,4S)-3-hydroxy-4-((2′-isopropyl-[1,1′-biphenyl]-4-yl)methoxy)pyrrolidine-2-carboxylic acid
(2R,3S,4S)-3-hydroxy-4-((2′-phenyl-[1,1′-biphenyl]-4-yl)methoxy)pyrrolidine-2-carboxylic acid
(2R,3S,4S)-3-hydroxy-4-((2′-trifluoromethyl-[1,1′-biphenyl]-4-yl)methoxy)pyrrolidine-2-carboxylic acid;
(2R,3S,4S)-3-hydroxy-4-((2-chlorol-[1,1′-biphenyl]-4-yl)methoxy)pyrrolidine-2-carboxylic acid;
(2R,3S,4S)-3-hydroxy-4-((2-fluoro-[1,1′-biphenyl]-4-yl)methoxy)pyrrolidine-2-carboxylic acid;
(2R,3S,4S)-3-hydroxy-4-((2-bromo-[1,1′-biphenyl]-4-yl)methoxy)pyrrolidine-2-carboxylic acid;
(2R,3S,4S)-3-hydroxy-4-((2-methyl-[1,1′-biphenyl]-4-yl)methoxy)pyrrolidine-2-carboxylic acid;
(2R,3S,4S)-3-hydroxy-4-((2-methoxy-[1,1′-biphenyl]-4-yl)methoxy)pyrrolidine-2-carboxylic acid;
(2R,3S,4S)-3-hydroxy-4-((2-isopropyl-[1,1′-biphenyl]-4-yl)methoxy)pyrrolidine-2-carboxylic acid;
(2R,3S,4S)-3-hydroxy-4-((2-phenyl-[1,1′-biphenyl]-4-yl)methoxy)pyrrolidine-2-carboxylic acid;
(2R,3S,4S)-3-hydroxy-4-((2-trifluoromethyl-[1,1′-biphenyl]-4-yl)methoxy)pyrrolidine-2-carboxylic acid;
(2R,3S,4S)-4-(biphenyl-4-ylmethoxy)-3-hydroxypyrrolidine-2-carboxylic acid;
(2S,3R,4R)-3-hydroxy-4-phenyl-proline;
(2S,3S,4R)-3-phenyl-4-hydroxy-proline;
(2S,3S,4R)-3-hydroxy-4-phenoxy-proline;
(2S,3S,4S)-3-phenoxy-4-hydroxy-proline;
Cis-4-methyl-trans-4-hydroxy-L-proline;
Cis-4-hydroxy-trans-4-methyl-L-proline;
Cis-3-methyl-trans-4-hydroxy-L-proline;
Cis-4-methyl-trans-2-hydroxy-L-proline;
cis-4-hydroxy-L-proline;
cis-4-isopropoxy-trans-2-hydroxy-L-proline;
trans-4-methoxy-cis-2-hydroxy-L-proline;
(2R,3S,4S)-3-hydroxy-4-[(3-phenoxybenzyl)oxy]pyrrolidine-2-carboxylic acid;
(2R,3S,4S)-3-hydroxy-4-[naphthalene-1-ylmethoxy]pyrrolidine-2-carboxylic acid;
Fluorenyl;
(2R,3S,4S)-4-([1,1′-biphenyl]-4-yl)-3-hydroxypyrrolidine-2-carboxylic acid;
(2R,3S,4S)-4-([1,1′-biphenyl]-2-yl)-3-hydroxypyrrolidine-2-carboxylic acid;
(2R,3S,4S)-4-([1,1′-biphenyl]-3-yl)-3-hydroxypyrrolidine-2-carboxylic acid; and
(2R,3S,4S)-4-(biphenyl-3-ylmethoxy)-3-hydroxypyrrolidine-2-carboxylic acid,
or a pharmaceutically acceptable salt thereof.

7. The method according to claim 5, wherein the pharmaceutical composition further comprises a pharmaceutically acceptable adjuvant, diluent or carrier.
